(19) United States
(12) Patent Application Publication
(10) Pub. No.: US 2024/0277797 A1
(43) Pub. Date: Aug. 22, 2024

(54) METHOD FOR TREATING OR PREVENTING NEOVASCULAR EYE DISEASE

(71) Applicant: Suzhou Mingren Pharmaceutical Biotechnology Co., Ltd., Suzhou (CN)

(72) Inventors: Jianwei ZHOU, Suzhou (CN); Zhan XIE, Suzhou (CN)

(21) Appl. No.: 18/650,002

(22) Filed: Apr. 29, 2024

Related U.S. Application Data
(63) Continuation-in-part of application No. PCT/CN2022/138732, filed on Dec. 13, 2022.

(30) Foreign Application Priority Data
Jun. 17, 2022 (CN) 202210689800.3

(51) Int. Cl. A61K 38/08; A61P 9/10; A61P 27/02

(52) U.S. Cl. CPC A61K 38/08; A61P 9/10; A61P 27/02

(57) ABSTRACT

A method for treating or preventing a neovascular eye disease including: administering a patient in need thereof a pharmaceutical composition including a polypeptide, the peptide having an amino acid sequence I or II; I: FPGSDRF (SEQ ID NO: 1)-Z; II: X-FPGSDRF (SEQ ID NO: 1)-Z; S represents phosphorylated serine; X and Z independently represents an amino acid or an amino acid sequence; X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉ (SEQ ID NO: 2), 6-aminohexanoic acid-(R)₉-F (SEQ ID NO: 3); and Z is selected from (G)n-RGD or A-(G)n-RGD (SEQ ID NO: 4), where n is an integer greater than or equal to 0, in the range of 0-10.

### CROSS-REFERENCE TO RELATED APPLICATIONS

This application is a continuation-in-part of International Patent Application No. PCT/CN2022/138732 with an international filing date of Dec. 13, 2022, designating the United States, now pending, and further claims foreign priority benefits to Chinese Patent Application No. 202210689800.3 filed Jun. 17, 2022. The contents of all of the aforementioned applications, including any intervening amendments thereto, are incorporated herein by reference.

### INCORPORATION-BY-REFERENCE OF SEQUENCE LISTING

This application contains a sequence listing, which has been submitted electronically in XML file and is incorporated herein by reference in its entirety. The XML file, created on Jan. 19, 2024, is named NJYK-00301-UUS.xml, and is 24,782 bytes in size.

### BACKGROUND

The disclosure relates to a method for treating or preventing a neovascular eye disease comprising administering a patient a polypeptide.

Neovascular ophthalmopathy refers to eye diseases that cause vision loss and irreversible damage to patients, and mainly include neovascular age-related macular degeneration (nAMD) and diabetic retinopathy (DR), retinal vein occlusion (RVO), neovascular glaucoma, and retinopathy of prematurity (ROP), etc.

Convectional first-line clinical treatment for neovascular eye diseases is intraocular injection of anti-vascular endothelial growth factor (VEGF) antibody drugs. Although anti-VEGF antibody drugs such as ranibizumab, aflibercept, and conbercept exhibit certain therapeutic effects, there are still some patients who have not achieved clinically significant visual improvement after treatment. Approximately 67.4% of nAMD patients are subjected to persistent vascular leakage in the macular area, and over 60% of nAMD patients experience poor visual recovery after 2 years of treatment. Due to the complexity of the conditions, some patients must receive long-term and repeated intraocular injections of anti VEGF drugs. Repeated intraocular injections cause heavy treatment pressure for patients and greatly increase the risk of complications, such as endophthalmitis, high intraocular pressure, and tearing of the retinal pigment epithelium.

### SUMMARY

To achieve the aforesaid objective, one objective of the disclosure is to provide a method for treating or preventing a neovascular eye disease, the method comprising: administering a patient in need thereof a pharmaceutical composition comprising a polypeptide, the peptide having an amino acid sequence I or II;
I: FPGSDRF (SEQ ID NO: 1)-Z;
II: X-FPGSDRF (SEQ ID NO: 1)-Z;
S represents phosphorylated serine;
X and Z independently represents an amino acid or an amino acid sequence;
X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉ (SEQ ID NO: 2), 6-aminohexanoic acid-(R)₉-F (SEQ ID NO: 3); and
Z is selected from (G)ₙ-RGD or A-(G)ₙ-RGD (SEQ ID NO: 4), where n is an integer greater than or equal to 0, in the range of 0-10.

In a class of this embodiment, the neovascular eye disease is wet macular degeneration.

In a class of this embodiment, the neovascular eye disease is neovascular age-related macular degeneration (nAMD).

In a class of this embodiment, the neovascular eye disease is diabetic retinopathy (DR).

In a class of this embodiment, the neovascular eye disease is retinal vein occlusion (RVO), neovascular glaucoma, or retinopathy of prematurity (ROP).

In a class of this embodiment, the polypeptide comprises an acetylated N-terminal and an amidated C-terminal.

In a class of this embodiment, the polypeptide has an amino acid sequence of FPGSDRF-RGD, and serine (S) of the amino acid sequence of FPGSDRF-RGD is phosphorylated.

In a class of this embodiment, the pharmaceutical composition further comprises a pharmaceutically acceptable excipient.

In a class of this embodiment, the pharmaceutical composition is for intraocular administration.

In a class of this embodiment, the pharmaceutical composition is for extraocular administration.

The following advantages are associated with the method for treating or preventing a neovascular eye disease of the disclosure:
The polypeptide of the disclosure can regulate reactive oxygen species (ROS)/NF-κB pathway in microglia to inhibit oxidative stress and inflammatory response, and on the other hand, the polypeptide can inhibit p-MEK1/2 and TRIM25, accelerate the degradation of SP1, and downregulate the transcription of integrins αVβ3 and MMP2 in endothelial cells, playing an anti-angiogenic role. Therefore, the polypeptide can be used as a candidate molecule for the treatment or prevention of neovascular eye disease, and can be used to prepare drugs for the treatment or prevention of neovascular eye disease.

### BRIEF DESCRIPTION OF THE DRAWINGS

FIGS. 1A-1G show the experimental results of Example 1 of the disclosure; FIG. 1A shows a technical roadmap for experimental model design scheme; FIG. 1B shows representative images of fluorescence angiography in choroidal neovascularization (CNV) mice 5 minutes after intraperitoneal injection of a contrast agent; FIG. 1C shows a comparison of scores of leakage intensity of mouse CNVs; FIG. 1D shows representative images of neovascularization in mouse choroidal slices after left ventricular perfusion with FITC-dextron; FIG. 1E shows statistical analysis of the area of mouse choroidal slices after left ventricular perfusion with FITC-dextron; FIG. 1F shows HE staining at the laser shooting point of CNV mice; and FIG. 1G shows analysis of relative thickness of lesions in CNV mice.

FIGS. 2A-2I show the experimental results of Example 2 of the disclosure; FIG. 2A shows immunostaining results of ROS at the lesions of CNV mice through intravitreal injection with 1 μL of PBS or JP1 40 μg (scale bar: 10 μm); FIG. 2B shows results of MDA, SOD and GPx activities in the choroidal tissues of CNV mice; FIG. 2C shows immunofluorescence results of IBA1 and Nrf2 in CNV mouse lesions, scale bar: 10 μm; FIG. 2D shows results of immunoblotting experimental analysis of Nrf2 in the choroidal tissues of CNV mice; FIG. 2E shows results of immunofluorescence experimental analysis of IBA1 and p-P65 in CNV mouse lesions, scale bar: 10 μm; FIG. 2F shows immunoblotting results of p-P65 and P65 in choroidal tissues of CNV mice; FIGS. 2G-2H shows immunofluorescence results of TNF-α (G) and IL-6 (H), scale bar: 10 μm; FIG. 2I shows immunoblotting results of TNF-α, IL-6 and VEGF in choroidal tissues of CNV mice treated with PBS or JP1.

FIGS. 3A-3I show the experimental results of Example 3 of the disclosure; FIG. 3A shows results of intracellular ROS levels in LPS treated BV2 cells with or without JP1 detected by DCFH-DA, scale: 20 μM; FIG. 3B shows quantitative analysis of the number of DCFH-DA positive cells detected by confocal microscopy; FIG. 3C shows immunofluorescence evaluation results of Nrf2 and IBA1 in BV2 cells, scale: 20 μM; FIG. 3D shows average fluorescence result of Nrf2 in BV2 cells; FIG. 3E shows immunoblot analysis results of Nrf2 in BV2 cells; FIG. 3F shows immunofluorescence detection results of p-P65 and IBA1 in BV2 cells, scale: 20 μM; FIG. 3G shows immunofluorescence detection results of TNF-α, IL-6, and iNOS in BV2 cells, scale: 20 μM; FIG. 3H shows the average fluorescence quantitative analysis results of p-P65, TNF-α, IL-6, and iNOS in BV2 cells; FIG. 3I shows Western blot analysis of p-P65, P65, VEGF, TNF-α and IL-6g in BV2 cells.

FIGS. 4A-4K show the experimental results of Example 4 of the disclosure; FIG. 4A shows the expression of integrins αV and β3 in the choroidal neovascular tissues of CNV mice after immunohistochemical staining; each pair of right pictures correspond to enlarged views of the areas in the small image boxes; FIG. 4B shows the protein content of p-MEK1/2, MEK1/2, SP1, integrins αV, β3, and CD31 in the choroidal tissues of CNV mice in the intravitreal injection of PBS group, the RBZ 10 μg group, the JP1 40 μg group, and the RBZ 5 μg+JP1 20 μg group through immunoblotting analysis; FIG. 4C shows the results of immunoblotting experiments of TRIM25 and MMP2 in the choroidal tissues of the four groups of CNV mice; FIGS. 4D-4I shows inhibition of tube formation (FIGS. 4D-4E), migration (FIGS. 4F-4G), and proliferation (FIGS. 4H-4I) of HUVECs by JP1; HUVECs were inhibited with VEGF (50 ng/mL) and different concentrations of JP1 (0, 50, 100, and 200 μM) for 24 h; FIG. 4D shows representative images of tube-forming assay; FIG. 4E shows numbers of closed tubes; FIG. 4F is the migratory ability of HUVECs detected by Transwell assay; FIG. 4G is the number of migrated cells; FIG. 4H is the EdU staining of HUVECs; FIG. 4I is the ratio of EdU-positive cells; FIGS. 4J-4K are the protein levees of p-MEK1/2, MEK1/2, SP1, integrin αV, β3, CD31, TRIM25 and MMP2 in VEGF and JP1-treated HUVECs through immunoblotting experiments. Note: Data in FIGS. 4E, 4G and 4I are expressed as mean±SEM. **p≤0.01, ***p≤ 0.001, with significant difference from cells in VEGF (50 ng/mL) treated group.

FIGS. 5A-5J show the experimental results of Example 5 of the disclosure; FIG. 5A shows a flow chart of the experimental procedure; FIG. 5B shows the results of retinal slices in 3-month diabetic group, RBZ group, JP1 group and a combined group of mice with FITC-dextron ventricular perfusion; FIG. 5C shows the results of the quantitative analysis of the retinal vascular density of the mice in each group with AngioTool software; FIG. 5D shows results of the quantitative analysis of the Evans blue leakage in the retinas of the mice in each group (n=6/group); FIG. 5E shows results of immunofluorescence analysis of IBA-1 and p-P65 in retinal tissues of diabetic mice in PBS- and JP1-treated groups; FIG. 5F shows results of immunoblot analysis of TNF-α, IL-6, and VEGF in retinal tissues of diabetic mice treated with PBS and JP1; FIG. 5G shows results of immunohistochemical analysis of p-P65, P65, VEGF, TNF-α and IL-6 protein levels in retina of diabetic mice treated with PBS and JP1; FIG. 5H shows results of immunohistochemical analysis of Occludin and ZO-1 in in retinal tissues of diabetic mice in the PBS and JP1-treated groups; FIG. 5I shows results of immunofluorescence experiments of the expression of CD31, Occludin and ZO-1 in the nerve fiber layer and ganglion cell layer of the retina of diabetic mice in the PBS and JP1-treated groups; FIG. 5J shows results of immunoblot experiments of Occludin and ZO-1 in layer of the retina of diabetic mice in PBS and JP1-treated groups. Note: Data in FIGS. 5C, 5D, 5F and 5H are expressed as mean±SEM. *P≤ 0.05, indicating difference with PBS group; **P≤0.01, indicating difference with PBS group; ***P≤0.001, indicating difference with PBS group; ****P≤0.0001.

FIGS. 6A-6H show the experimental results of Example 6 of the disclosure; FIG. 6A is a flow chart of an experimental process of a CNV mouse model with intraperitoneal injection of JP1; FIG. 6B shows representative fundus fluorescein angiography (FFA) images and quantitative analysis results of vascular leakage in CNV mice injected with JP1 intraperitoneally; FIG. 6C shows typical images of FITC-dextron labeled blood vessels on choroidal tiling plates and quantitative results of fluorescent vascular regions through ImageJ software; FIG. 6D shows results of H&E staining and quantitative analysis of relative thickness of CNV lesion; FIG. 6E shows a flow chart of the experimental process of CNV mouse models injected intraperitoneally with FITC-JP1 and FITC; FIG. 6F shows representative FFA images and quantitative analysis results of fluorescence intensity of CNV lesions in FITC-JP1 and FITC alone groups; FIGS. 6G-6H show representative fluorescence images of stretched preparations of choroid at designated time points after intraperitoneal injection in two groups (FITC-JP1 5 mg vs FITC alone 0.99 mg). Note: The data in FIGS. 6B, 6C, 6D and 6F are represented by the mean±SEM. O. N: Optic nerve. I. P: Intraperitoneal injection. ns, no difference with the JP1 (1 mg, I.P.) group, ***P≤0.001, compared with the JP1 (1 mg, I.P.) group.

FIG. 7 is a schematic diagram of the mechanism of action of JP1 in the conclusion part of the disclosure.

FIGS. 8A-8E show JP1-RGD eye drops can effectively reduce CNV leakage and area; FIG. 8A is an experimental flow diagram of JP1-RGD eye drops for treating a CNV mouse model; FIG. 8B shows representative FFA images of JP1-RGD eye drops for treating vascular leakage in CNV mice; FIG. 8C shows a quantitative analysis result of vascular leakage of JP1-RGD eye drops for treating vascular leakage in CNV mice; FIG. 8D shows typical images of CD31 labeled blood vessels on choroidal plain films, where the scale is 50 μM; FIG. 8E shows quantitative analysis of fluorescent vascular area through ImageJ software; the data in FIGS. 8C and 8E are represented by mean±SEM; **P≤0.01, ***P≤0.001, ****P≤0.0001, all of which are differences compared to the control group. CNV: choroidal neovascularization, RBZ: Ranibizumab; IVT: intravitreal.

FIGS. 9A-9C show fluorescence distribution of FITC-JP1-RGD eye drops in the fundus of CNV mice; FIG. 9A shows fluorescence images of live CNV mice in the 5 mM FITC-JP1-RGD group and 0.99 mM FITC group at different times after eye drops; FIG. 9B shows a fluorescence intensity-time curve at the lesion site of CNV, and the data is expressed by mean±SEM; FIG. 9C shows specific aggregation of FITC-JP1-RGD at CNV lesions of the mice in the 5 mM FITC-JP1-RGD group after 0.5 hours' eye drops through frozen sections; the white box in FIG. 9C indicates the CNV lesion area.

### DETAILED DESCRIPTION

To further illustrate the disclosure, embodiments detailing a method for treating or preventing a neovascular eye disease are described below. The materials, methods, and experimental conditions used in each example are attached after each embodiment. Unless otherwise stated, the materials and experimental methods used are conventional materials and conventional experimental methods.

### Example 1

This example shows that JP1 inhibits 532 nm laser-induced choroidal neovascularization in mice. The sequence of JP1 is FPGSDRF-RGD, and the amino acid serine (S) is phosphorylated.

Choroidal neovascularization (CNV) is the most common pathological process of wet macular degeneration. 532 nm laser-induced CNV mouse model is widely used in the study of wet macular degeneration mechanism and drug efficacy. Anti-vascular endothelial growth factor (VEGF) drugs, such as Ranibizumab, have been widely used as first-line agents in clinical trials and therapy. To evaluate the effect of JP1 on choroidal neovascularization in vivo, a 532-nm laser-induced CNV mouse model was constructed and Ranibizumab was used as a positive control drug.

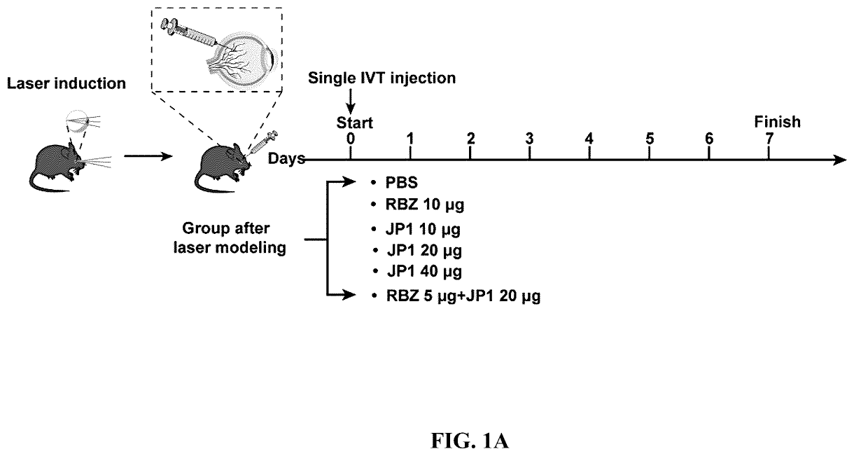

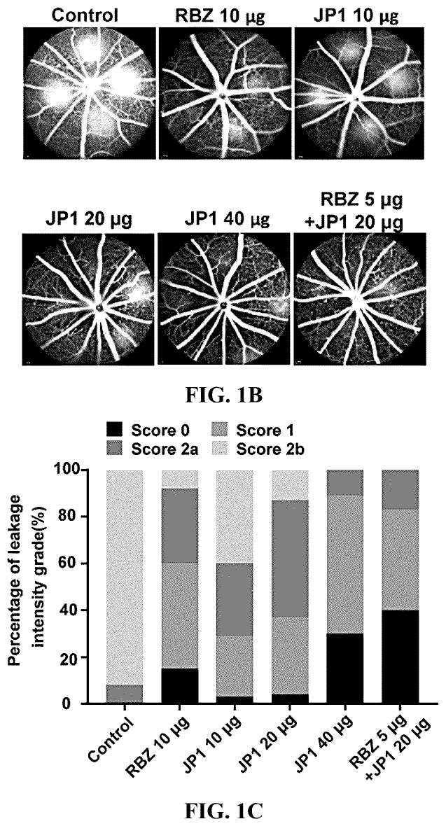

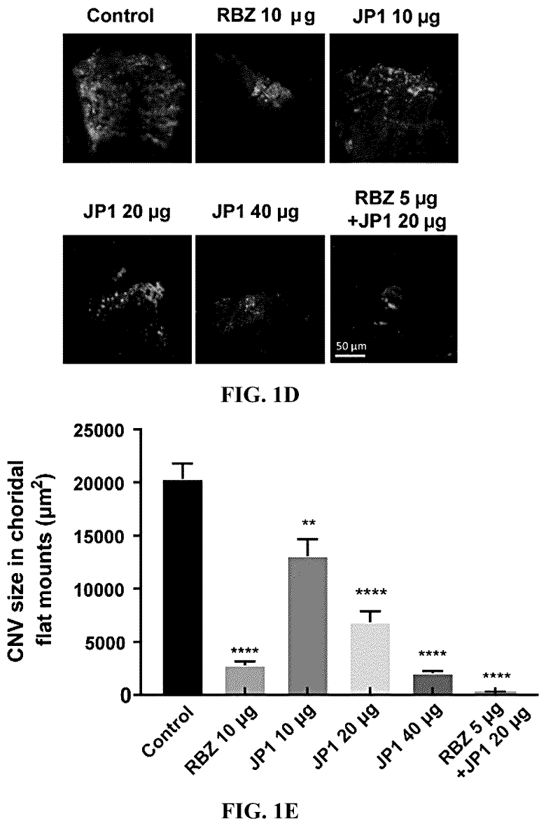

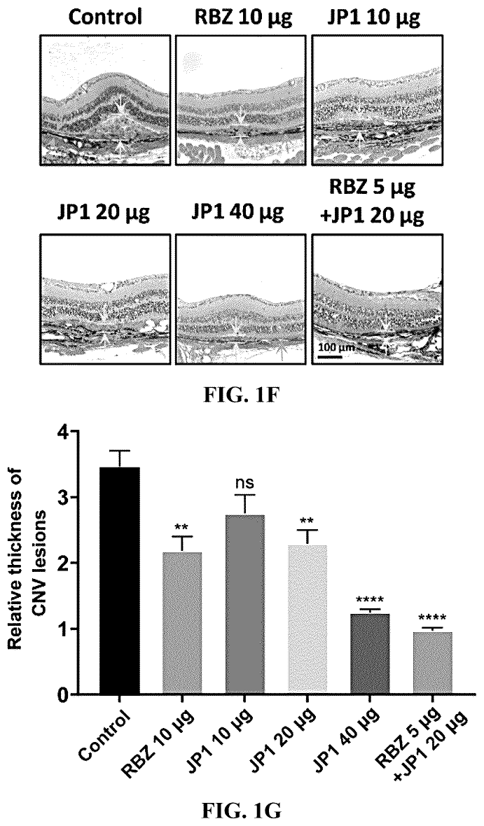

The results are shown in FIG. 1. The results showed intravitreal injection of JP1 inhibited 532-nm laser-induced CNV in a dose-dependent manner (FIGS. 1A-1G). In the JP1-treated group, the percentage of CNV lesions increased according to the 0 and 1 leakage scores, whereas the percentage of CNV lesions decreased according to the 2a and 2b leakage scores (FIG. 1C). Choroidal pavements and immunofluorescence staining of FITC-dextron left ventricle perfusion showed that intravitreal injection of JP1 significantly reduced the area of laser-induced CNV as compared to the control group (FIGS. 1D-1E). The efficacy of medium-dose JP1 (20 μg) was similar to that of Ranibizumab (10 μg), and high-dose JP1 (40 μg) had a more potent inhibitory effect on CNV than Ranibizumab (10 μg) (FIGS. 1E and 1G). At one week after laser induction, low, medium, and high doses of JP1 effectively inhibited laser-induced CNV (FIGS. 1C, 1E, 1G). Notably, the combination of JP1 20 μg+Ranibizumab 5 μg produced a synergistic effect with better efficacy than either single-agent use (FIGS. 1E and 1G). These results showed that compared with the clinical classic therapeutic drug Ranibizumab, intravitreal injection of 40 μg JP1 could effectively inhibit CNV, and the intervention of JP1 in combination with a halved dose of Ranibizumab was even more effective.

### Example 2

This example aims to alleviate oxidative stress and inflammation of microglia in a CNV mouse model using JP1.

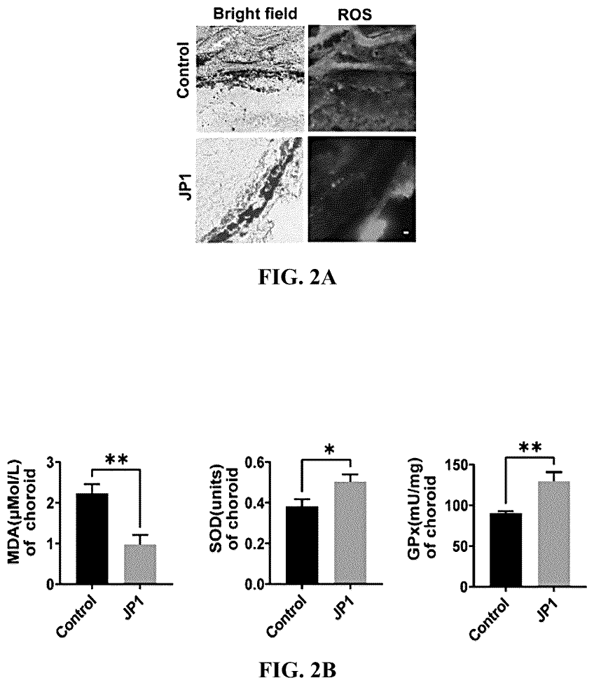

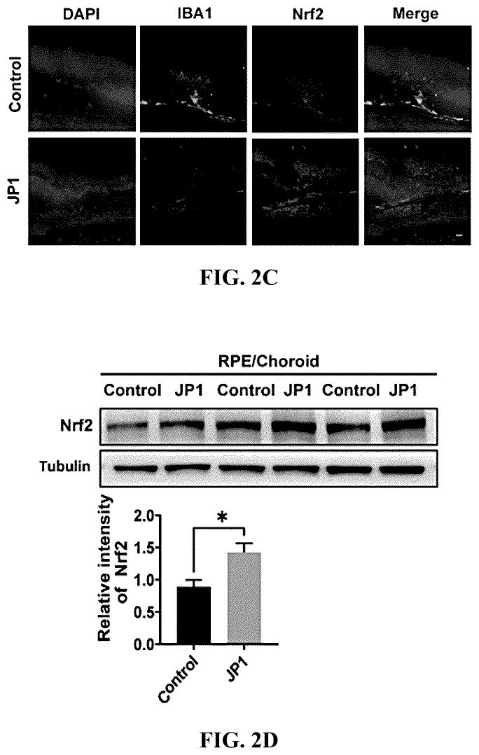

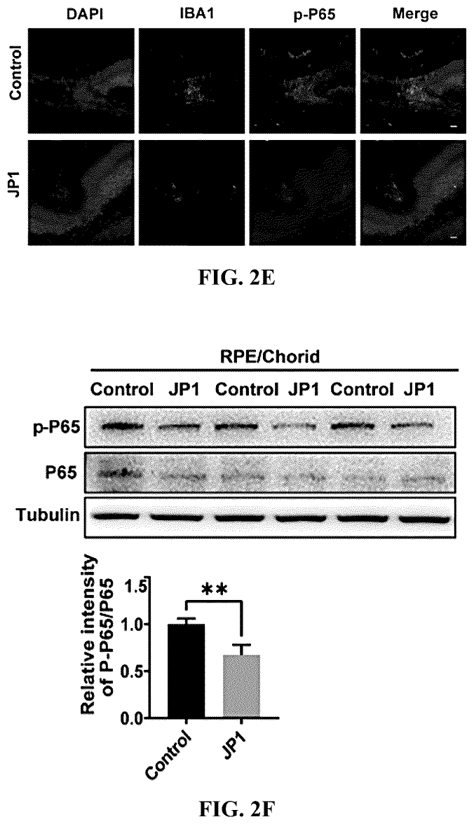

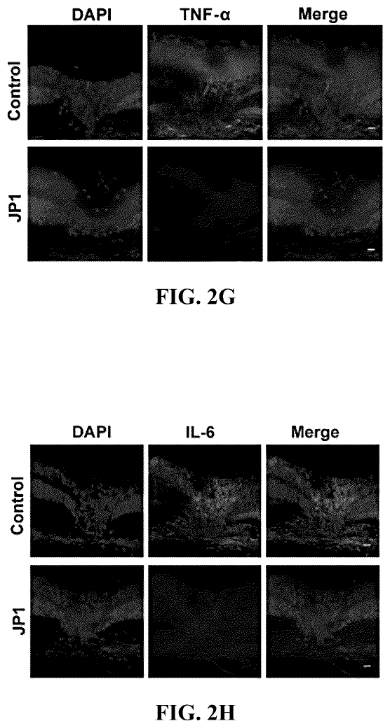

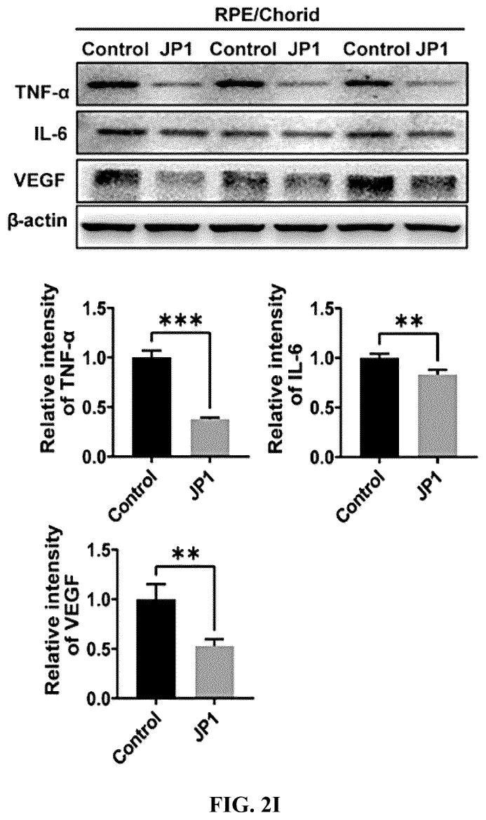

Oxidative stress and inflammation are two important pathological events in the occurrence and development of nAMD. To evaluate the level of oxidative stress damage in the lesion area of CNV mice, the levels of reactive oxygen species (ROS) were measured from choroidal tissue slices of CNV mice (FIG. 2A). Compared with the control group, the fluorescence intensity of ROS in the CNV slices of JP1 treated eyes was significantly reduced (FIG. 2A). In addition, the levels of antioxidant enzymes, including superoxide dismutase (SOD) and glutathione peroxidase (GPx), significantly increased in the choroidal tissue of JP1 treated eyes, while the levels of malondialdehyde (MDA), a marker of oxidative stress, significantly decreased (FIG. 2B)

Previous studies have shown that JWA activates the expression of nuclear factor E2-related factor 2 (Nrf2), inhibits the production of ROS, and enhances the resistance of neuronal cells to paraquat-induced neurotoxicity. Lot of evidence shows that microglia, the resident immune cells of the retina, play an important role in neovascular eye diseases such as nAMD and DR. Modulation of microglia reactivity is emerging as a promising therapeutic strategy for neovascular eye diseases. Therefore, the effect of JP1 on the level of microglial oxidative stress damage was further investigated. During immunofluorescence staining, IBA1 was used to label microglia. Double staining for IBA-1 and Nrf2 showed that JP1 significantly reduced the aggregation activation of IBA-1-positive microglial cells and enhanced the fluorescence intensity of Nrf2 in choroidal tissue (FIG. 2C). Immunoblotting experiments showed that Nrf2 protein expression was significantly increased in choroidal tissues of CNV mice after JP1 treatment (FIG. 2D).

During the development of nAMD, oxidative stress can induce inflammation. Considering NF-κB signaling pathway is closely related to ROS, the regulation of JP1 on the NF-κB signaling pathways and downstream inflammatory factors was detected in vivo and in vitro using a laser induced CNV mouse model and mouse microglia BV2.

Compared with the control group, the immune reactivity of p-P65 at the CNV lesion in eye tissue slices treated with JP1 was reduced (FIG. 2E). In addition, immunoblotting experiments showed that JP1 downregulated the phosphorylation level of P65 in the choroidal tissue of CNV mice (FIG. 2F). Furthermore, the expression of two classical pro-inflammatory factors (TNF-α and IL-6) was assayed at the CNV lesions. The results of immunofluorescence staining (FIGS. 2G-2H) and immunoblotting experiment (FIG. 2I) showed that TNF-α and IL-6 expression were significantly down-regulated in JP1-treated eyes, while VEGF protein expression was also reduced (FIG. 2I).

### Example 3

This example shows that JP1 reduces lipopolysaccharide induced oxidative stress and inflammatory response in BV2 cells.

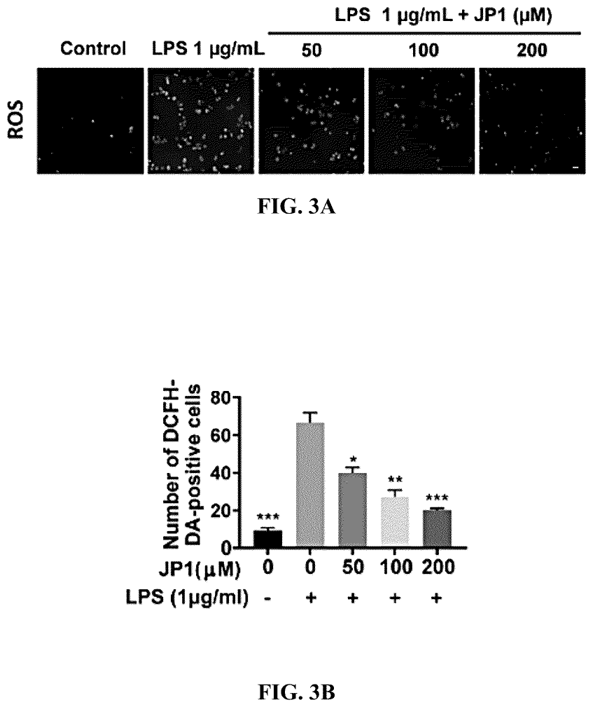

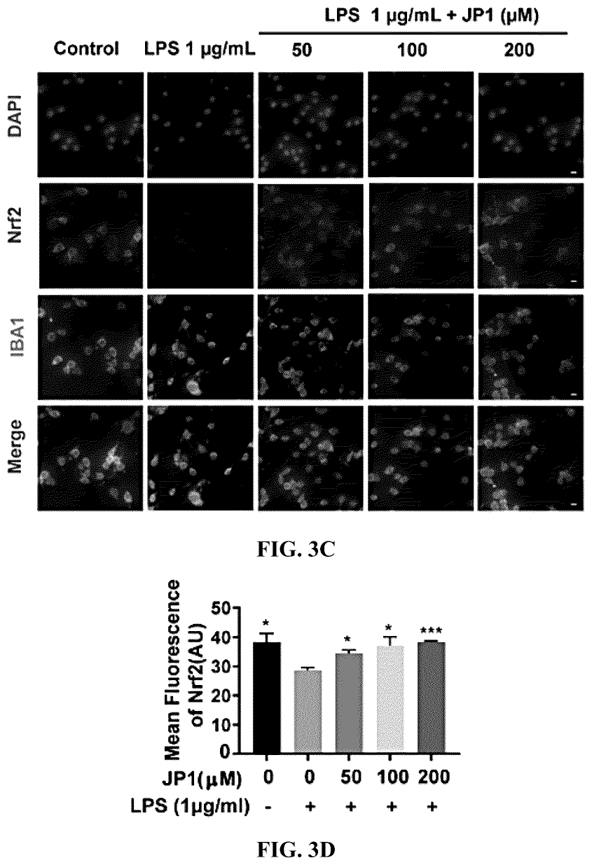

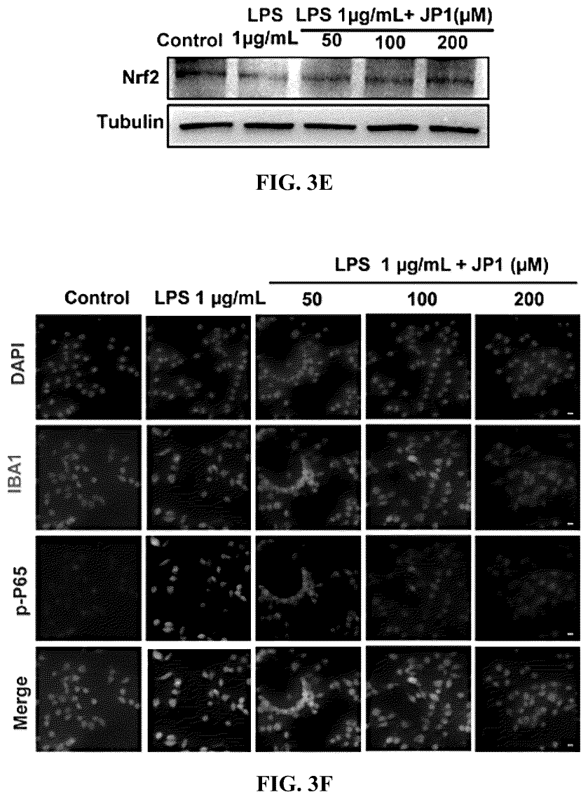

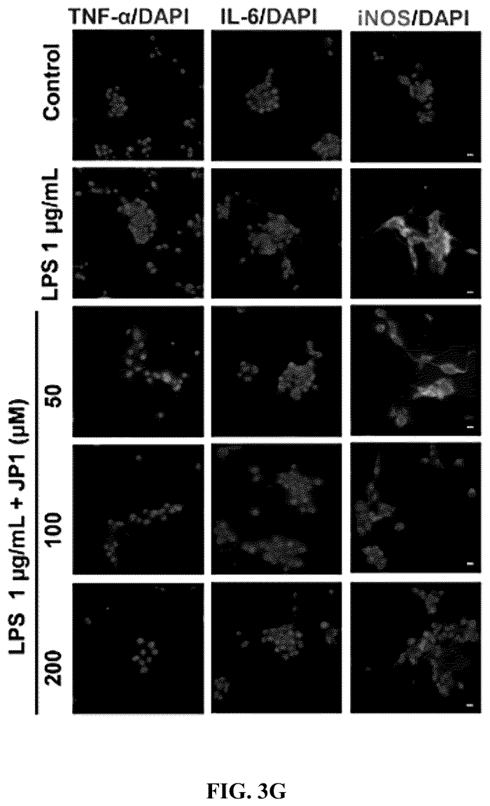

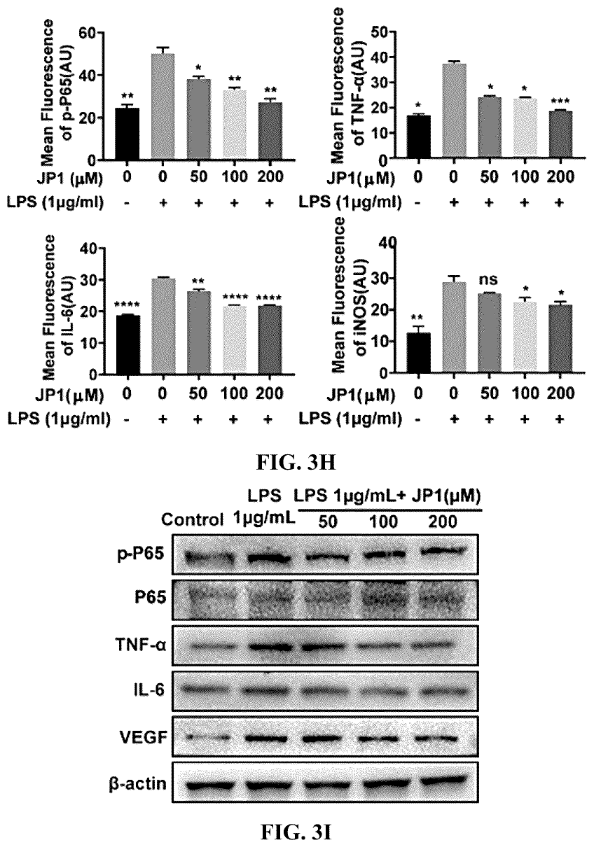

To further validate the regulatory mechanism of JP1 on oxidative stress and inflammation in microglia, lipopolysaccharide (LPS, 1 μg/mL) and JP1 (0, 50, 100, and 200 μM) were used to treat mouse microglia (BV2) cells for 24 h, and to observe the effects of JP1 on BV2 cells. Fluorescence quantitative analysis was used to evaluate the ROS levels in BV2 cells (FIG. 3A). The results showed a significant increase in ROS in microglia after LPS treatment for 24 h (FIGS. 3A-3B). The production of ROS in BV2 cells was negatively correlated with the JP1 concentration level (FIGS. 3A-3B). In addition, immunofluorescence staining FIGS. 3C-3D) and immunoblotting experiments (FIG. 3E) showed that JP1 dose-dependently up-regulated Nrf2 levels in BV2 cells, which is consistent with the existing literature of JWA. Thereafter, the anti-inflammatory potential of JP1 was detected using the LPS-treated BV2 cell model. Immunofluorescence staining showed that in BV2 cells, LPS upregulated the expression levels of p-P65 and inflammatory factors (TNF-α, IL-6 and iNOS), while JP1 dose-dependently downregulated the expression levels of p-P65 and inflammatory factors (FIGS. 3F-3H). Immunoblotting assay analysis further confirmed that 1000 ng/mL LPS treatment for 24 h activated the NF-κB pathway with up-regulation of inflammatory factors (VEGF, TNF-α, and IL-6), whereas JP1 inhibited the phosphorylation of p-P65 in a dose-dependent manner and effectively reduced the levels of inflammatory factors (FIG. 3I).

The results show that JP1 attenuates microglial oxidative stress damage and inflammatory responses by modulating the ROS/NF-κB signaling pathway.

### Example 4

This example shows that JP1 inhibits angiogenesis by modulating the MEK1/2/SP1/integrin αVβ3 axis and TRIM25/SP1/MMP2 axis in vascular endothelial cells.

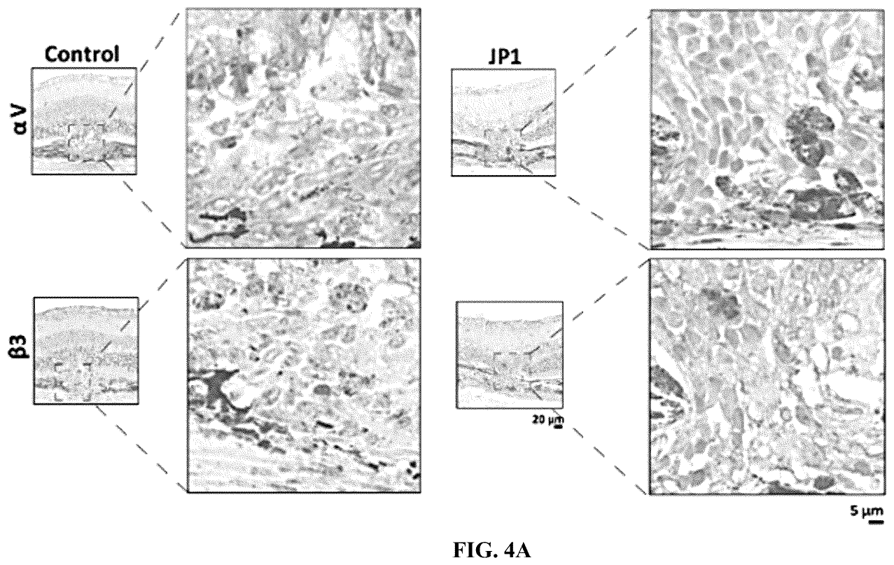

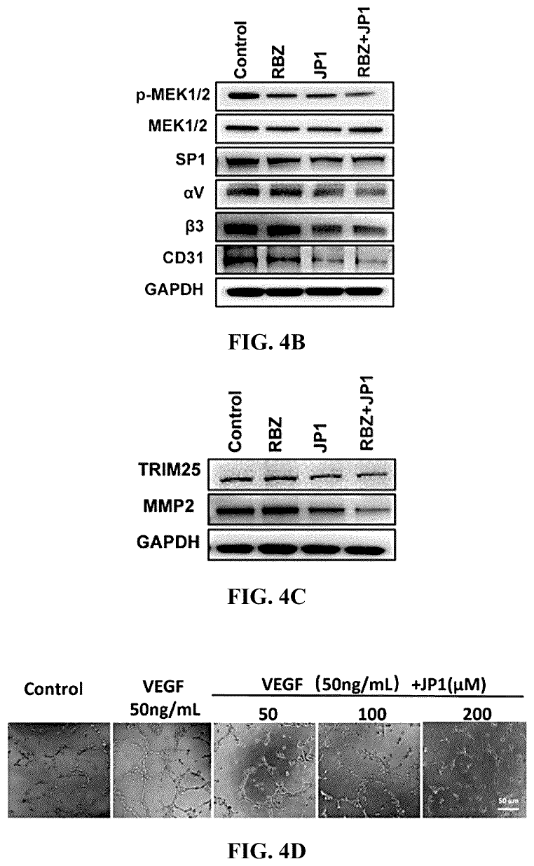

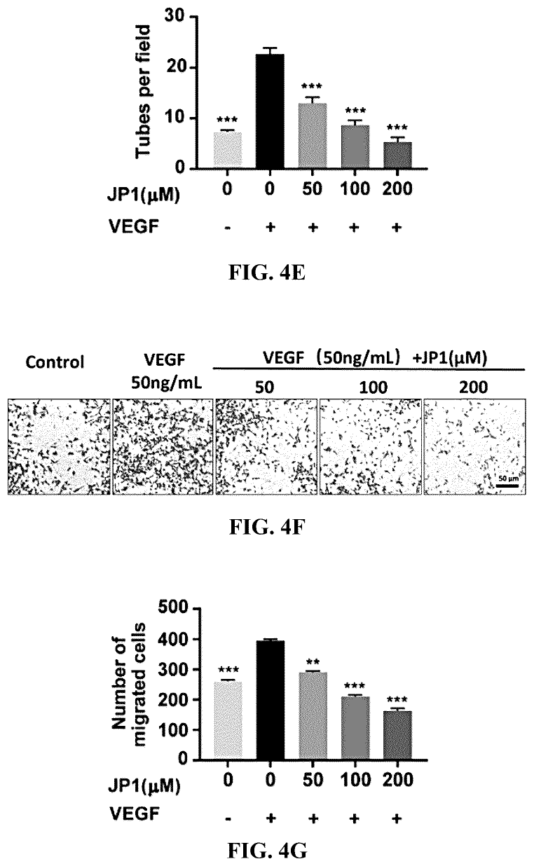

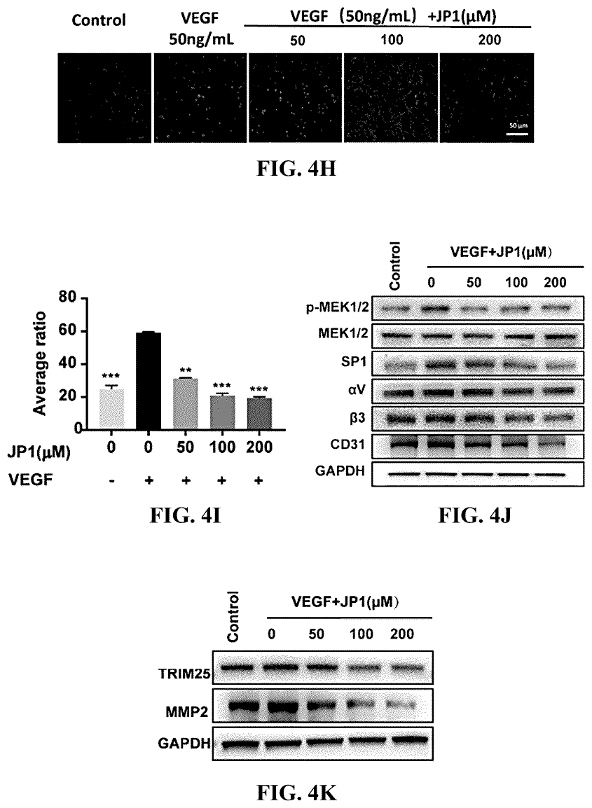

JP1 is a functional peptide designed based on the functional fragment of JWA protein. Therefore, suppose that the inhibition mechanism of CNV by JP1 is similar to the anti-cancer mechanism of JWA gene in gastric cancer and melanoma, and then the hypothesis is verified by in vivo and in vitro experiments. Previous literature showed that JP1 inhibited melanoma by regulating the MEK1/2/SP1/integrin αVβ3 axis. Therefore, the expression of integrin αV and β3 in CNV lesion tissues was firstly examined. Immunohistochemistry showed the expression of the integrin αV and β3 was downregulated at JP1-treated ocular lesions (FIG. 4A). Thereafter, the expression of MEK1/2, p-MEK1/2, SP1, integrin αV, β3, and CD31 was detected in the choroidal tissues of CNV mice by immunoblotting experiments. As shown in FIG. 4B, JP1 inhibited the phosphorylation of MEK 1/2 and the expression of SP1, and down-regulated the protein expression levels of integrin αV, β3, and CD31 in the choroidal tissues of CNV mice (FIG. 4B). It was also detected that compared to the positive control agent RGZ, JP1 down-regulated the protein expression levels of TRIM25 and MMP2 in the choroidal tissues of CNV mice (FIG. 4C). To further validate the inhibition mechanism of JP1 on angiogenesis, human umbilical vein vascular endothelial cells (HUVECs) were analyzed by Transwell assay, EdU assay, tube formation assay, and immunoblotting assay. HUVEC cells were incubated with VEGF (50 ng/ml) and different concentrations of JP1 (0, 50, 100, 200 μM) for 24 h. The results showed that JP1 dose-dependently inhibited VEGF-induced angiogenesis (FIGS. 4D-4E), migration (FIGS. 4F-4G), and proliferation of HUVEC cells (FIGS. 4H-4I), and JP1 regulated MEK1/2-SP1-Integrin αvβ3 and TRIM25/SP1/MMP2 axis (FIGS. 4J-4K) in a dose-dependent manner, which is consistent with the in vivo experiments.

### Example 5

This example shows that JP1 can partially attenuate the disruption of the blood-retinal barrier in diabetic mice by regulating NF-κB signaling pathway of microglia cells.

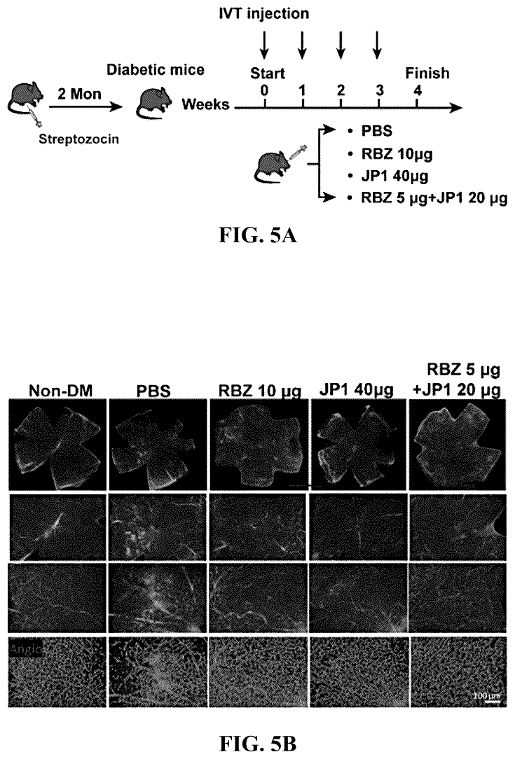

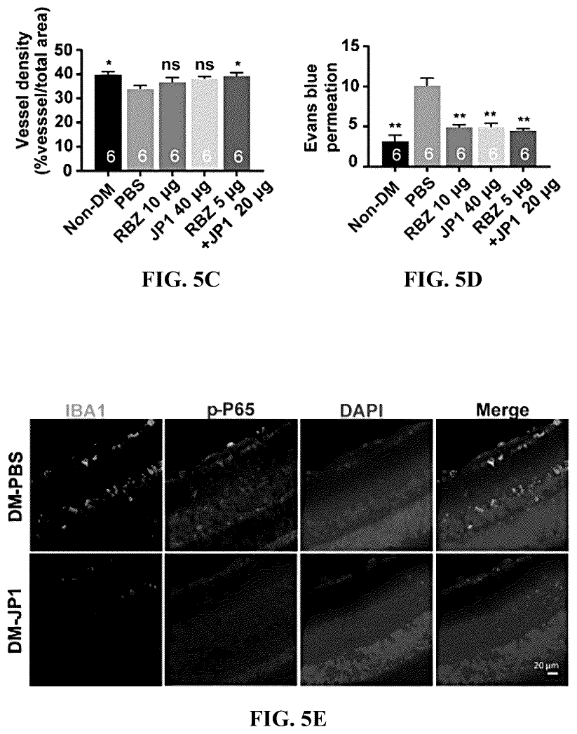

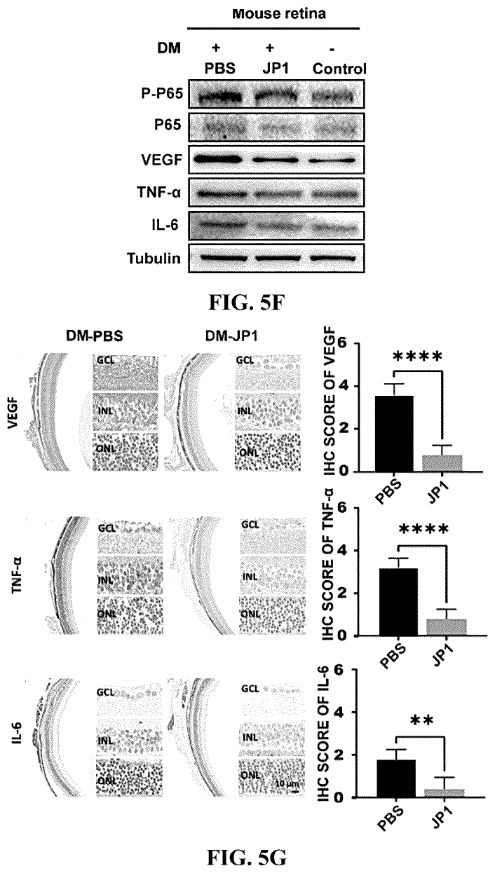

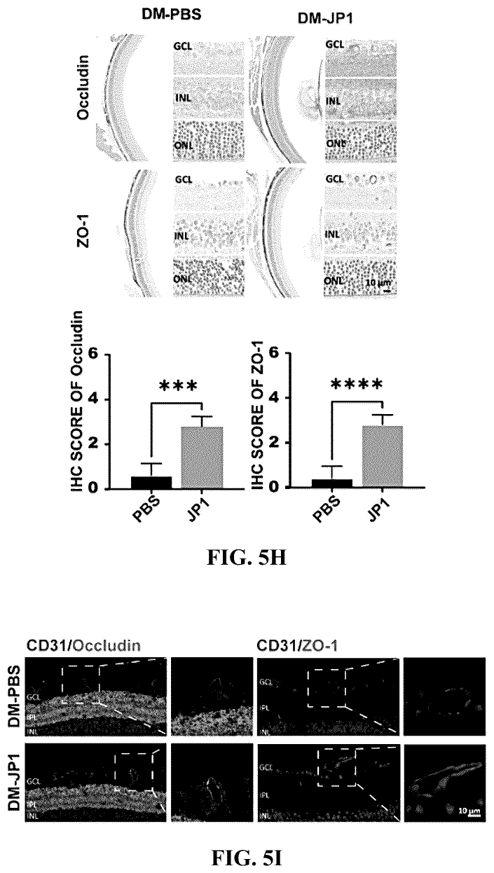

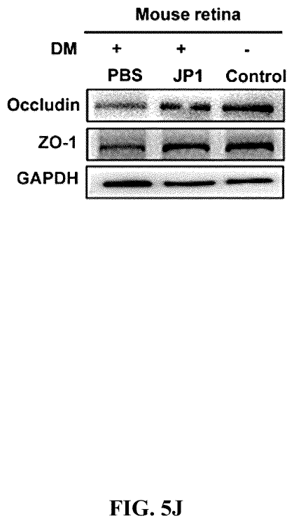

Diabetes retinopathy is a main cause of blindness in working age people. To study the therapeutic effect of JP1 on diabetes retinopathy, a streptozotocin (STZ)-induced diabetes mouse model was constructed, which is a classic model for clinical research and drug experiments. Stretched preparation of retina of FITC-Dextron left ventricular perfusion showed that three months after the course of diabetes, PBS group mice had obvious vascular leakage around the optic disc and around the retina, accompanied by retinal vascular curvature, and no perfusion area increased (FIG. 5B). In addition, the retinal vascular density in the PBS treatment group significantly decreased compared to normal mice of the same age, but there was no significant decrease in retinal vascular density in the RBZ or JP1 treatment groups of mice (FIG. 5C). Furthermore, the Evans blue experiment showed significant retinal vascular leakage in the PBS treated group of mice, while no significant retinal leakage was observed in the JP1 treated group (FIG. 5D). The above results suggest that JP1 has inhibitory effect on vascular leakage in diabetes mice.

Inflammation is a main pathological feature of diabetic retinopathy (DR). Studies have shown that activation of microglia in DR can release inflammatory factors to activate the NF-κB signaling pathway. Existing research results showed that the JWA gene alleviates neuroinflammation by regulating the NF-κB signaling pathway, thereby exerting a neuroprotective effect against dopamine neuron degeneration. Therefore, the example continued to investigate whether JP1 could regulate the NF-κB signaling pathway and attenuate STZ-induced disruption of the blood retinal barrier (BRB) in diabetic mice (FIG. 5A). Immunofluorescence experiments showed that the fluorescence intensity of p-P65 and IBA1 was reduced in the retina of JP1-treated mice compared with the PBS group (FIG. 5E). Immunoblot experiments showed that TNF-α, IL-6 and VEGF were downregulated in the retinal tissues of JP1-treated diabetic mice (FIG. 5F). Immunohistochemistry assay analysis further confirmed that JP1 inhibited the NF-κB signaling pathway and downregulated inflammatory factors (VEGF, TNF-α, and IL-6) (FIG. 5G). In addition, immunohistochemistry (FIG. 5H), immunofluorescence (FIG. 5I), and immunoblotting assay analysis (FIG. 5J) showed that JP1 attenuated the loss of retinal tight junction proteins (Occludin and ZO-1) in diabetic mice.

### Example 6

This example relates to intraperitoneal injection of JP1 to effectively reduce CNV leakage and area.

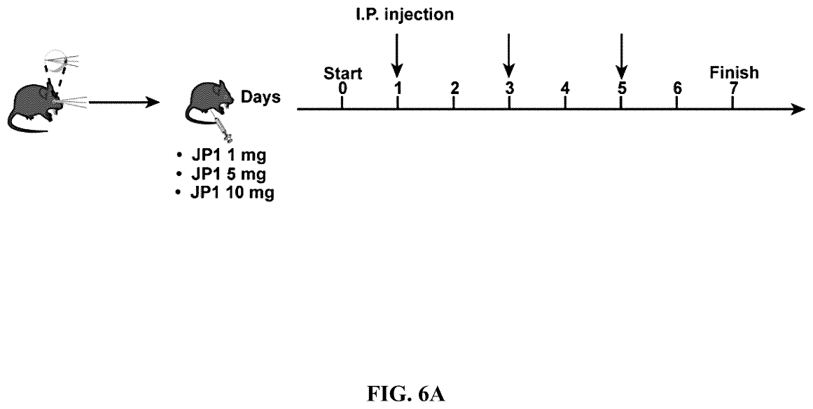

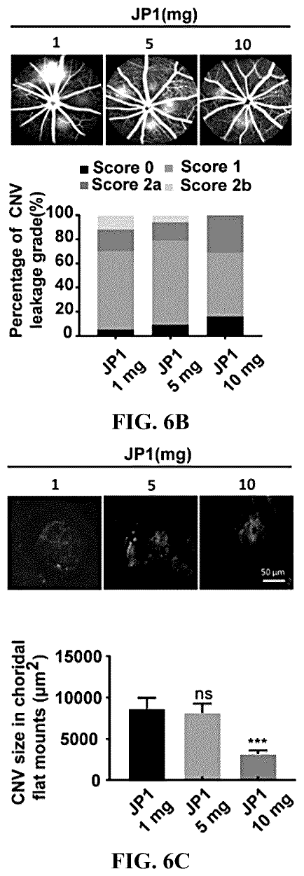

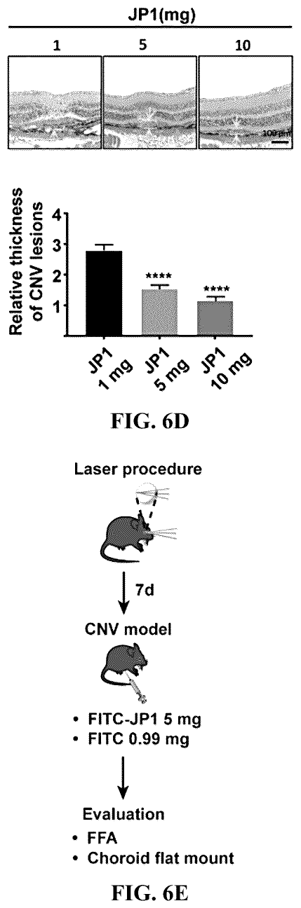

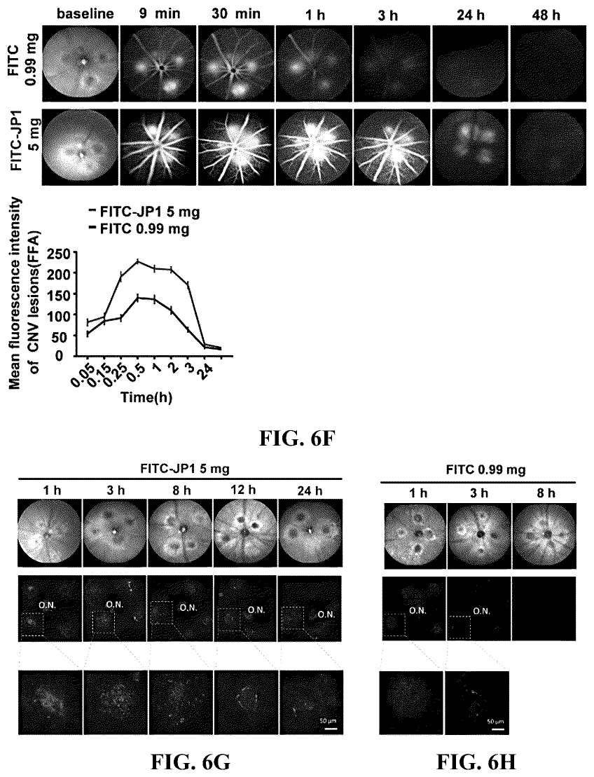

Integrin αVβ3 is overexpressed in tumor cells and activated endothelial cells. The identification of the integrin of Arg-Gly-Asp (RGD) sequence has been specifically studied as therapeutic targets for tumors. JP1 is a peptide that connects the RGD sequence and specifically targets integrin αVβ3. It is speculated that JP1 has the potential to break through the blood eye barrier and target CNV lesions through extraocular administration to exert therapeutic effects. This example explores the therapeutic potential of JP1 extraocular administration (intraperitoneal administration) in a laser induced CNV mouse model (FIG. 6A). The level of vascular leakage (FIG. 6B), average area of CNV lesions (FIG. 6C), and relative thickness (FIG. 6D) indicate that intraperitoneal administration of JP1 inhibits CNV in a dose-dependent manner. In the laser induced CNV model, FFA and fluorescence microscopy imaging were performed after intraperitoneal injection of FITC-JP1 and FITC at specified time points (FIG. 6E). The CNV lesions in the FITC-JP1 group still showed fluorescence at 24 hours, while no fluorescence was observed in the FITC alone group (FIG. 6F). The fluorescence intensity in the FITC-JP1 group was higher than that in the FITC alone group (FIG. 6G). Retinal choroidal plain films confirmed that JP1 enhanced the accumulation of FITC at CNV lesions (FIG. 6H), confirming the targeting effect of JP1 on CNV lesions. The above results indicate that intraperitoneal injection of JP1 can effectively reduce CNV vascular leakage and area.

### Example 7

This example aims to verify the effect of JWA peptides other than JP1 in fighting against neovascular eye diseases.

The example uses the JWA peptides shown in the table below for experiments according to Examples 1 to 6, and the amino acid serine of each JWA peptide is phosphorylated.

| No. | Sequence | No. | Sequence | No. | Sequence |
|---|---|---|---|---|---|
| JP2 | FPGSDRF-G- | JP3 | FPGSDRF-(G)₄- | JP4 | FPGSDRF-(G)₁₀- |
| | RGD (SEQ ID | | RGD(SEQ ID | | RGD(SEQ ID |
| | NO: 5) | | NO: 6) | | NO: 7) |
| JP5 | FPGSDRF-A- | JP6 | FPGSDRF-A-G- | JP7 | FPGSDRF-A- |
| | RGD(SEQ ID | | RGD(SEQ ID | | (G)₄-RGD(SEQ |
| | NO: 8) | | NO: 9) | | ID NO: 10) |
| JP8 | FPGSDRF-A- | JP9 | FFPGSDRF- | JP10 | FFPGSDRF-G- |
| | (G)₁₀-RGD(SEQ | | RGD(SEQ ID | | RGD(SEQ ID |
| | ID NO: 11) | | NO: 12) | | NO: 13) |
| JP11 | FFPGSDRF-(G)₄- | JP12 | FFPGSDRF-(G)₁₀- | JP13 | FFPGSDRF-A- |
| | RGD(SEQ ID | | RGD(SEQ ID | | RGD(SEQ ID |
| | NO: 14) | | NO: 15) | | NO: 16) |
| JP14 | FFPGSDRF-A-G- | JP15 | FFPGSDRF-A- | JP16 | (R)₉-FPGSDRF- |
| | RGD(SEQ ID | | (G)₁₀-RGD(SEQ | | RGD(SEQ ID |
| | NO: 17) | | ID NO: 18) | | NO: 19) |
| JP17 | (R)₉-FPGSDRF- | JP18 | (R)₉-FPGSDRF-A- | JP19 | (R)₉-FPGSDRF-A- |
| | (G)₁₀-RGD(SEQ | | RGD(SEQ ID | | (G)₁₀-RGD(SEQ |
| | ID NO: 20) | | NO: 21) | | ID NO: 22) |
| JP20 | (R)₉-F-FPGSDRF- | JP21 | (R)₉-F-FPGSDRF- | JP22 | (R)₉-F-FPGSDRF- |
| | RGD(SEQ ID | | (G)₁₀-RGD(SEQ | | A-RGD(SEQ ID |
| | NO: 23) | | ID NO: 24) | | NO: 25) |
| JP23 | (R)₉-F-FPGSDRF- | JP24 | 6-aminocaproic | JP25 | 6-aminocaproic |
| | A-(G)₁₀- | | acid-FPGSDRF- | | acid-FPGSDRF- |
| | RGD(SEQ ID | | RGD(SEQ ID | | (G)₁₀-RGD(SEQ |
| | NO: 26) | | NO: 27) | | ID NO: 7) |
| JP26 | 6-aminocaproic | JP27 | 6-aminocaproic | JP28 | 6-aminocaproic |
| | acid-FPGSDRF-A- | | acid-FPGSDRF-A- | | acid-F-FPGSDRF- |
| | RGD(SEQ ID | | (G)₁₀-RGD(SEQ | | RGD(SEQ ID |
| | NO: 8) | | ID NO: 11) | | NO: 12) |
| JP29 | 6-aminocaproic | JP30 | 6-aminocaproic | JP31 | 6-aminocaproic |
| | acid-F-FPGSDRF- | | acid-F-FPGSDRF- | | acid-F-FPGSDRF- |
| | (G)₁₀-RGD(SEQ | | A-RGD(SEQ ID | | A-(G)₁₀- |
| | ID NO: 15) | | NO: 16) | | RGD(SEQ ID |
| | | | | | NO: 18) |
| JP32 | 6-aminocaproic | JP33 | 6-aminocaproic | JP34 | 6-aminocaproic |
| | acid-(R)₉- | | acid-(R)₉- | | acid-(R)₉- |
| | FPGSDRF- | | FPGSDRF-(G)₁₀- | | FPGSDRF-A- |
| | RGD(SEQ ID | | RGD(SEQ ID | | RGD(SEQ ID |
| | NO: 19) | | NO: 20) | | NO: 21) |
| JP35 | 6-aminocaproic | JP36 | 6-aminocaproic | JP37 | 6-aminocaproic |
| | acid-(R)₉- | | acid-(R)₉-F- | | acid-(R)₉-F- |
| | FPGSDRF-A- | | FPGSDRF- | | FPGSDRF-(G)₁₀- |
| | (G)₁₀-RGD(SEQ | | RGD(SEQ ID | | RGD(SEQ ID |
| | ID NO: 22) | | NO: 23) | | NO: 24) |
| JP38 | 6-aminocaproic | JP39 | 6-aminocaproic | | |
| | acid-(R)₉-F- | | acid-(R)₉-F- | | |
| | FPGSDRF-A- | | FPGSDRF-A- | | |
| | RGD(SEQ ID | | (G)₁₀-RGD(SEQ | | |
| | NO: 25) | | ID NO: 26) | | |

Limited by description space, detailed experimental data is not listed in this example. The obtained experimental data shows that the detection results of the JWA peptides according to Examples 1 to 6 are basically consistent with that for JP1.

### Example 8

JP1-RGD (same as JP1 in aforesaid examples) peptide eye drops for the treatment of choroidal neovascularization in a mouse model

Choroidal neovascularization (CNV) mouse model: After sufficient abdominal anesthesia in C57BL/6 mice, topicamide dilated the pupils and sodium hyaluronate was applied to the cornea for protection. Place a circular cover glass (diameter 8 mm) on sodium hyaluronate. The right retina of the mouse received 532 nm laser photocoagulation, with a laser energy setting of 250 mW, a laser spot size of 50 μm, and a laser burst time of 100 ms. Through the fundus laser machine, it is possible to shoot once in the 3:00, 6:00, 9:00, and 12:00 areas within a distance of about 1 PD from the mouse optic nerve. At the moment of laser emission, bubbles formed under the subretinal pigment layer can be observed, indicating that the Bruce membrane has been broken. At the same time, laser spots under the retina are also observed, with a grayish white color.

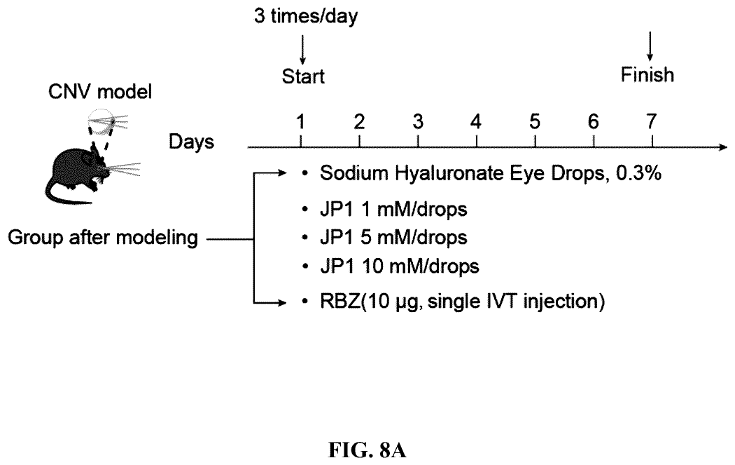

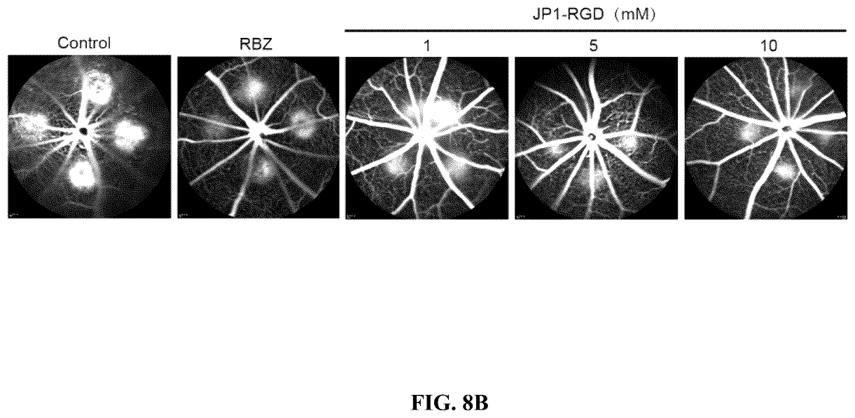

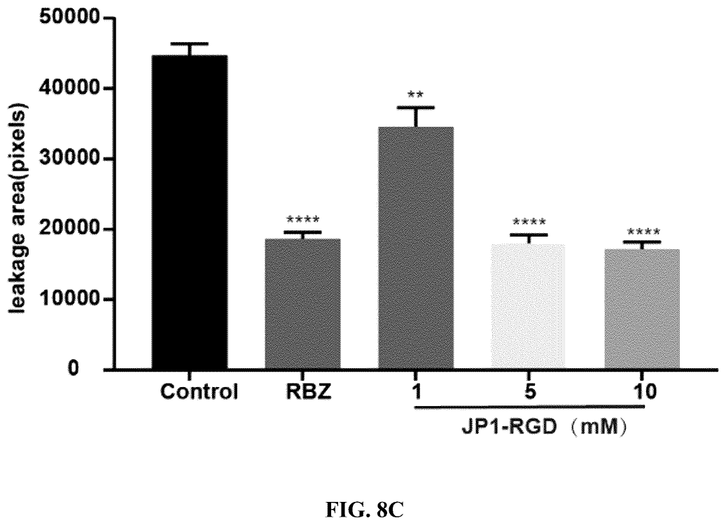

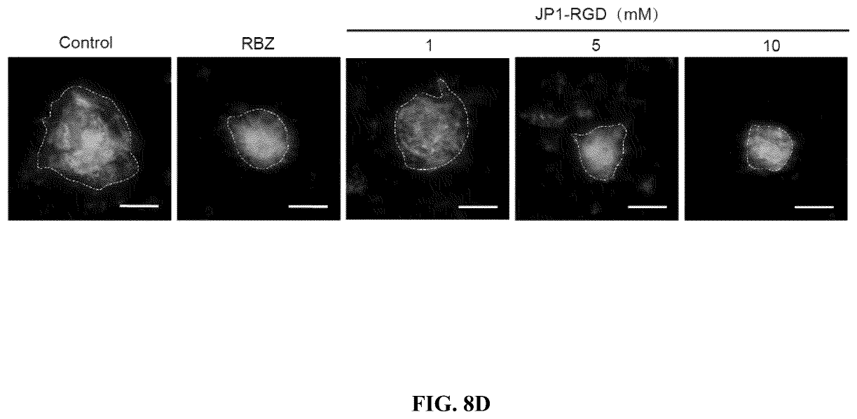

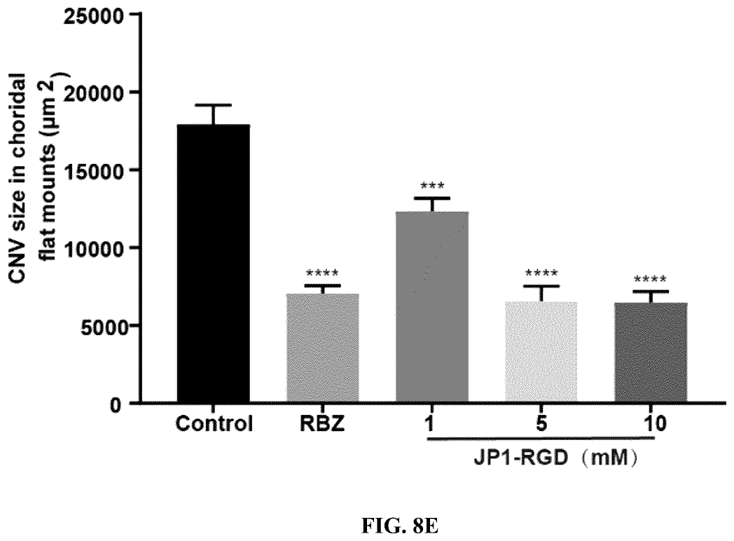

FIG. 8A is an experimental flow diagram of JP1-RGD (same as JP1 in aforesaid examples) eye drops for treating a CNV mouse model. Specifically, after modeling with 532 nm laser, 50 C57BL/6 mice were randomly divided into 5 groups, with 10 mice in each group and 10 eyes. The 5 groups were a surgical solvent control group (0.3% sodium hyaluronate eye drops), a JP1 1 mM group, a JP1 5 mM group, a JP1 10 mM group, and a positive drug group of ranibizumab (RBZ). After laser modelling, the RBZ group was immediately injected into the vitreous cavity (10 μL). Interventional drug eye drops were used 3 times a day for a total of 1 week. FIG. 8B shows representative FFA images of JP1-RGD eye drops for treating vascular leakage in CNV mice; FIG. 8C shows a quantitative analysis result of vascular leakage of JP1-RGD eye drops for treating vascular leakage in CNV mice; FIG. 8D shows typical images of CD31 labeled blood vessels on choroidal plain films, where the scale is 50 μM; FIG. 8E shows quantitative analysis of fluorescent vascular area through ImageJ software; the data in FIGS. 8C and 8E are represented by mean±SEM; **P≤0.01, ***P≤0.001, ****P≤0.0001, all of which are differences compared to the control group. CNV: choroidal neovascularization, RBZ: Ranibizumab; IVT: intravitreal.

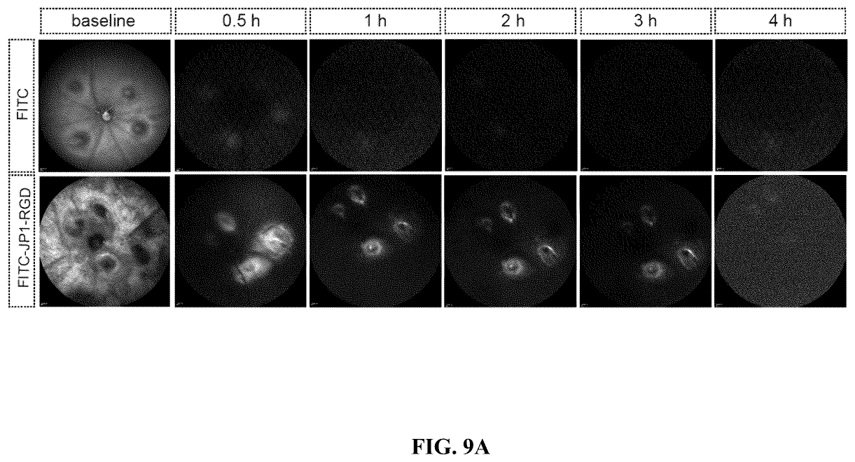

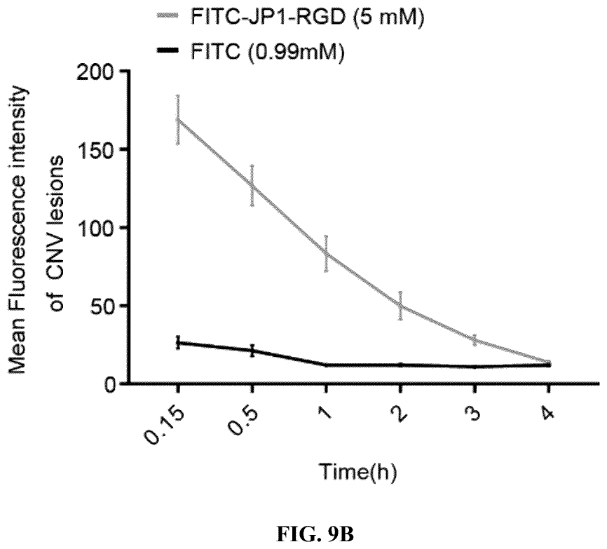

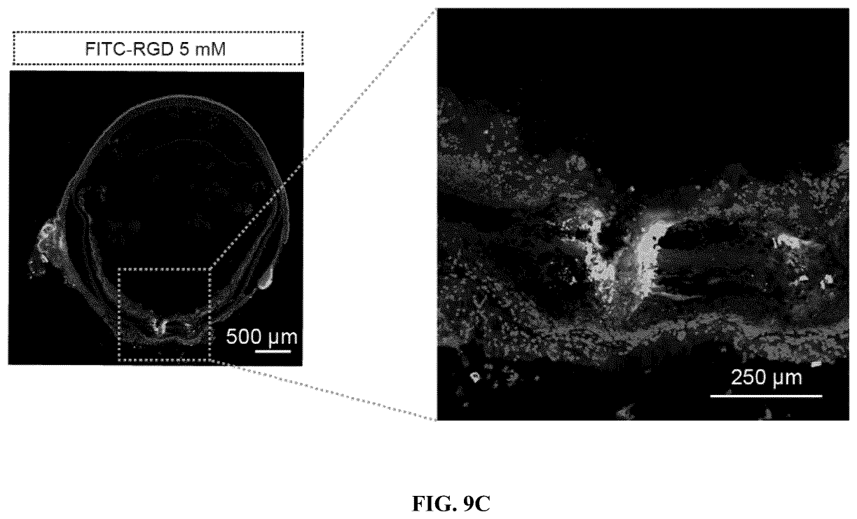

FIGS. 9A-9C show fluorescence distribution of FITC-JP1-RGD (same as FITC-JP1 in aforesaid examples) eye drops in the fundus of CNV mice; FIG. 9A shows fluorescence images of live CNV mice in the 5 mM FITC-JP1-RGD group and 0.99 mM FITC group at different times after eye drops; FIG. 9B shows a fluorescence intensity-time curve at the lesion site of CNV, and the data is expressed by mean±SEM; FIG. 9C shows specific aggregation of FITC-JP1-RGD at CNV lesions of the mice in the 5 mM FITC-JP1-RGD group after eye drops of 0.5 hours through frozen sections; the white box in FIG. 9C indicates the CNV lesion area.

### Conclusion

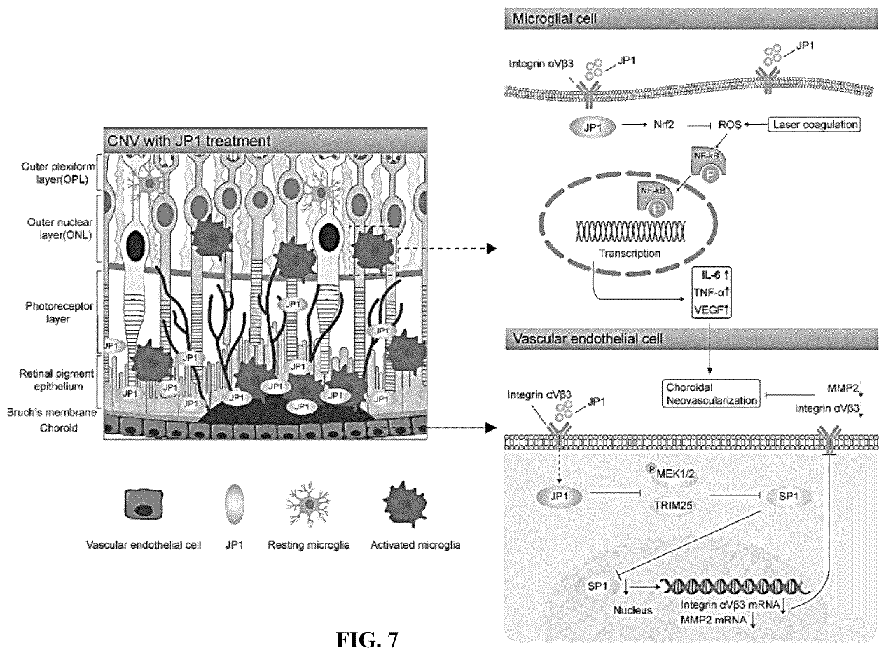

As can be seen from the above examples, the disclosure confirms the therapeutic effect of a series of JWA polypeptides represented by JP1 on choroidal neovascularization in a 532-nm laser-induced CNV mouse model and retinal vascular leakage in a streptozotocin-induced diabetic mouse model. On the one hand, these JWA peptides inhibited oxidative stress and inflammatory responses by modulating the microglial ROS/NF-κB pathway; on the other hand, these JWA peptides exerted anti-neovascularization effects by inhibiting p-MEK1/2 and TRIM25, accelerating SP1 degradation, and down-regulating the transcription of integrin αvβ3 and MMP2 in the vascular endothelial cells (FIG. 7). Therefore, these peptides could be promising as candidate molecules for the preparation of drugs for treatment or prevention of neovascular eye diseases.

The materials, methods, experimental model conditions, etc., used in each of the above examples are shown below:

### I. Main Instruments

Finesse ME+Paraffin Slicer, Thermo, USA;
RS-232 type CO₂ incubator, HERA CELL, Germany;
Pannoramic SCAN slice scanner, 3DHISTECH, Hungary;
Wet Electrotransferometer, Bio-Rad, USA;
LQ-A2003 Electronic Balance, Shanghai Yaoxin Electronic Technology Co.;
Waterpro PS Ultrapure Water System, Labconco, USA;
CK40 Inverted optical microscope, OLYMPUS, Japan;
DS-U3 Digital Photographic System, Nikon, Japan;
CYRO-2000 Liquid Nitrogen Tank, CBS, USA;
X-30R Ultracentrifuge, BECKMAN, USA;
Diagenode Bioruptor Non-contact Ultrasonic Breaker, Diagenode, Belgium;
CL-17R Centrifuge, Thermo Scientific, USA;
Infinite M200 PRO Multifunctional Enzyme Labeler, TECAN, Switzerland;
PAC-300 Voltage Regulator, Bio-Rad, USA;
125-BR Vertical Electrophoresis Instrument, Bio-Rad, USA;
Chemiluminescent Gel Imager, Bio-Rad, USA;
Corresponding Volume Microsampler, Eppendorf, Germany;
GI54T Vertical Automatic Pressure Steam Sterilizer, Zhiwei (Xiamen) Instrument Co.;
THZ-C Constant Temperature Oscillator, Jiangsu Taicang Experimental Instrument Factory;
PF03 Ventilated Cabinet, Guangzhou Pan American Industrial Co.;
−20° C. refrigerator, Haier, China;
−80° ° C. refrigerator, Thermo Scientific;
DK-600 Electrothermal constant temperature water tank, Shanghai Jinghong Experimental Equipment Co.;
Zeiss LSM 510 Laser confocal microscope, Carl Zeiss, Germany;
Oven, DHG-9140A, Shanghai Huitai Instrument Manufacturing Co.;
Embedding machine, Wuhan Junjie Electronics Co.;
Freezing table, Wuhan Junjie Electronics Co.;
Tissue Spreader, Zhejiang Jinhua Kedi Instrument Co.;
Coverslip, 10212432C, Jiangsu Shitai Experimental Equipment Co.;
Microwave Oven, P70D20TL-P4, Glance Microwave Oven & Appliance Co.;
Microscope, Nikon E100;
Orthostatic fluorescence microscope, NIKON ECLIPSE C1, Japan;
Fundus Fluorescence Angiography Machine, Carl Zeiss, Germany;
Fundus laser machine, Carl Zeiss, Germany;
Pathology Slicer, RM2016, Leica Instruments (Shanghai) Co.; Fundus camera, Carl Zeiss, Germany;
Hamiliton Micro-injector, Bonaduz, Switzerland;
Imaging System Nikon DS-U3, Japan.

### II. Cell Sources and Culture Conditions

Human umbilical vein vascular endothelial cells (HUVECs) were purchased from the Shanghai Institute of Biochemistry and Cell Biology, Chinese Academy of Sciences (Shanghai, China). rmii-1640 (Thermo Scientific) with 10% fetal bovine serum (FBS; Gibco) and 1% streptomycin and penicillin (Gibco) was added to the medium of the HUVECs cells. Cells were cultured in an intermittently humidified incubator at 37° C. with 5% CO₂. HUVECs were treated with VEGF (50 ng/ml) for 24 h in the presence of JP1 (0, 50, 100, 200 μM) treatment.

Immortalized mouse microglia (BV2) were gifted by Prof. Han Feng from the Department of Clinical Medicine, College of Pharmacy, Nanjing Medical University, and used in generation 7. BV-2 cell culture medium was supplemented with 10% fetal bovine serum, 1% penicillin/streptomycin, and 1% GlutaMAX (Gibco) in DMEM/F12 (Biosharp). Cells were cultured in a humidified incubator at 37° ° C. with 5% CO₂. BV2 cells were treated with lipopolysaccharide (LPS) 1000 ng/mL) in the presence of JP1 (0, 50, 100, 200 μM) treatment for 24 h.

### III. Animal Origin

The experimental mice were obtained from Shanghai Lingchang Co., Ltd. and were male, aged six to eight weeks old, and raised in the Laboratory Animal Center of Nanjing Medical University. All experimental mice and experimental animal operations were approved by the Ethics Committee of Nanjing Medical University under the ethical number IACUC-1811067, and the mice were raised at a temperature of 18-22° C. and a humidity of 50% to 60%. The waterer was 250 mL in volume, and water was given in a drinking bottle, and the water was changed two to three times a week. The bedding was changed twice a week. The mice were anesthetized with 2.5% chloral hydrate+5% urethane (0.5 g urethane dissolved in 10 mL of 2.5% chloral hydrate) by intraperitoneal injection at a dosage of 0.1 mL/10 g. Mydrin eye drops (Santen Pharmaceutical Co., Ltd., Osaka, Japan) were used to dilate the pupils; and the surface of the eyeballs was coated with sodium hyaluronate, a medical substance, to protect the cornea.

### IV. Main Reagents

### 1 Experimental Peptides

Peptide JP1 and others were synthesized by GL Biochem (Shanghai) Ltd. and Hybio Pharmaceutical Co., Ltd. (Shenzhen, China, under standard GMP conditions. Purity >98%, water-soluble. Freeze dried powder is stored for a long time at −20° C.

### 2 Main Reagents

Cell culture medium DMEM/F12 (China, Lanjieke Technology Co., Ltd.), DMEM (U.S., GIBCO); fetal bovine serum (China, Hangzhou Sijiqing Biological Co., Ltd.); penicillin, streptomycin, ciprofloxacin (China, Shandong Qilu Pharmaceutical Co., Ltd.); DAPI, BCA Protein Concentration Determination Kit (China, Shanghai Biyuntian Biotech Co., Ltd.), ECL color development solution (U.S., Cell Signaling Technology, Inc.).

### 3 Experimental Antibodies

Antibodies used in immunofluorescence experiments: iba1 (012-26723, 1:100, WAKO), anti-Nrf2 (16396-1-AP, 1:500, Proteintech), anti-TNF-α (ab183218, 1:100, Abcam), anti-IL-6 (bs-6309R, 1:100. Bioss), anti-CD31 (sc-376764, 1:100, Santa Cruz), phospho-NF-κBp65 (Ser536, 1:1600, Cell Signaling Technology), anti-Occludin (27260-1-AP, 1:1600, Proteintech), anti-Occludin (27260-1-AP, 1:1600, Proteintech), anti-TNF-α (ab183218, 1:100, Abcam), anti-IL-6 (bs-6309R, 1:100 Proteintech), anti-ZO-1 (21773-1-AP, 1:4000, Proteintech).

Antibodies used in immunohistochemistry experiments: anti-αV (ab179475, 1:500, Abcam), anti-β3 (13166s, 1:250, Cell Signaling Technology), anti-TNF-α (60291-1-Ig, 1:1000, Proteintech), anti-IL-6 (bs-0782R, 1:500, Bioss) and anti-VEGF (sc-53462, 1:500, Santa).

Antibodies used in immunoblotting experiments: anti-CD31 (sc-376764, 1:100, Santa), anti-αV: (ab179475, 1:5000, Abcam), anti-β3 (4702s, 1:1000, Cell Signaling Technology), anti-MMP2 (18309-1-AP, 1:1000, Proteintech), anti-TRIM25 (12573-1-AP, 1:1000, Proteintech), anti-MEK1/2 (1:1000, Cell Signaling Technology), anti-P-MEK1/2 (Ser217/221, 1:1000, Cell Signaling), anti-SP1 (21962-1-AP, 1:1000, Proteintech), anti-VEGF (sc-53462, 1:200, Santa), anti-NF-κBp65 (66535-1-Ig, 1:1000, Proteintech), Phospho-NF-κBp65 (Ser536, 1:1000, Proteintech), anti-Occludin (27260-1-AP, 1:1000, Proteintech), anti-ZO-1 (21773-1-AP, 1: Anti-TNF-α (17590-1-AP, 1:1000, Proteintech), anti-IL-6 (bs-0782R, 1:1000, Bioss), anti-B-actin (AF0003, 1:1000, Beyotime), anti-GAPDH (AF5009, 1:1000, Beyotime), anti-Tubulin (AT819, 1:1000, Beyotime), anti-Tubulin (AT819, 1:1000, Beyotime).

### V. Reagent Preparation

### 1. *Preparation of Immunoblotting and Immunoprecipitation Reagents

1) Upper gel buffer: stored at room temperature, the standard formula is 3.0 g Tris+ddH₂O, with a total volume of 1 L, adjust the pH to 6.8.
2) Lower gel buffer: stored at room temperature, the standard formula is 18.15 g Tris+ddH₂O, with a total volume of 1 L, adjust the pH to 8.8.
3) Electrophoresis buffer: the standard formula is 14.40 g glycine+3.03 g Tris-base+1 g SDS+ddH₂O, with a total volume of 1 L, instantly prepared when needed.
4) Wet transfer solution: instantly prepared when needed, the formula is 5.81 g Tris+2.92 g glycine+ddH₂O, with a total volume of 800 mL, and then add 200 mL of methanol to 1 L.
5) 10×TBS buffer: 24.2 g Tris and 80 g NaCl dissolved in 800 mL of ddH₂O, adjust the pH to 7.6, 10-fold dilution, and 1 mL of Tween 2.0 was added.
6) Blocking solution: 10 g skimmed milk powder was dissolved in 1×TBS, with a total volume of 200 mL.
7) Elution solution: add 15 g of glycine, 1 g of SDS and 10 mL of Tween 2.0 into 1 L of ddH₂O, adjust the pH to 2.2, stored in a dark place at 4° C.

### 2. Preparation of Immunohistochemistry Reagents

1) 4% paraformaldehyde (PFA): 4 g of PFA powder was fully dissolved in 100 mL of PBS solution, and filtered for use.
2) 95% ethanol: 190 mL of anhydrous ethanol was added to a measuring cylinder, and ddH₂O was added to the measuring cylinder, until a total volume was 200 mL.
3) 80% ethanol: 160 mL of anhydrous ethanol was added to a measuring cylinder, and ddH₂O was added to the measuring cylinder, until a total volume was 200 mL.
4) 70% ethanol: 140 mL of anhydrous ethanol was added to a measuring cylinder, and ddH₂O was added to the measuring cylinder, until a total volume was 200 mL.
5) PBST: a mixture of 1 L ddH₂O+7 g Na₂HPO₄−12 H₂O+0.5 g NaH₂PO₄-2H₂O+9 g NaCl was dissolved in 1000 mL of water.

### 3. Preparation of General Anesthetics for Small Animals

1) Mouse: 2.5% chloral hydrate+5% urethane, the initial dose was 0.1 mL/20 g; 0.2 mL was administered intraperitoneally to adult mice in good condition. The amount of anesthetics in elderly mice or mice with diabetes should be reduced as appropriate. Formula: 10 mL NS+0.25 g hydrated chloraldehyde+0.5 g urethane.
2) Rabbits: 20% urethane, initial dose: 10 mL/2 kg, 12.5 mL/2.5 kg, 15 mL/3 kg. Formula: 10 mL NS+2 g urethane. Rabbits were shortly anesthetized, 4 mL of isoflurane was sprayed on a sterile gauze and placed about 20 seconds in front of the rabbits' nose. Before the rabbit lost consciousness, there was usually a provocation, followed by satisfactory anesthesia.

### VI. Experimental Model and Conditions

### 1. A Mouse Model of Choroidal Neovascularization Induced by 532 nm Laser

After C57BL/6 mice were adequately anesthetized intraperitoneally, the pupils were dilated with tropicamide, and sodium hyaluronate was applied to the cornea for protection. A wash free circular cover glass (8 mm in diameter) was placed on top of the sodium hyaluronate. The retinas of both eyes were subjected to 532 nm laser photocoagulation with a laser energy of 250 mW, a laser spot size of 50 μm, and a laser burst time of 100 ms. A fundus laser machine fired once in each of the 3:00, 6:00, 9:00, and 12:00 areas within a distance of approximately 1 PD from the mouse optic nerve, with visible laser spots at the shooting site. At the moment of laser emission, bubbles formed beneath the pigment layer of the retina were observed, indicating that the Bruch membrane had ruptured. At the same time, laser spots under the retina were also observed, with a grayish white color. After laser modeling, the intraocular injection group was immediately injected with intervention drugs into the mouse vitreous cavity. Under a stereomicroscope, a gauze was placed under the head of the mouse, and the head was adjusted to be parallel to the eyeball plane and the tabletop. The corneal surface was coated with viscoelastic agent, and water was added to the viscoelastic agent to form a smooth mirror surface. A 33 G insulin needle was used to create a hole about 1 mm behind the mouse corneal edge. A 33 G Hamilton (2.5 uL syringe, 10 mm pointed injection needle) injection needle was inserted in the hole, and 1 uL intervention drug was rapidly injected. The needle was stopped for 5 seconds and quickly withdrawn. Erythromycin eye ointment was applied to mouse eyes, and make sure to completely cover the cornea when applying the ointment. The vitreous cavity administration group was randomly divided into 6 groups immediately after laser treatment, with each group receiving 1 uL of intervention drug through intravitreal injection. The intervention drugs were PBS, Ranibizumab 10 μg, JP1 10 μg, JP1 20 μg, JP1 40 μg, and JP1 20 μg+Ranibizumab 5 μg, respectively. The intraperitoneal injection group was injected with low, medium, and high concentrations of JP1 (1 mg, 5 mg, and 10 mg, 100 uL) on the second day after laser treatment, once every two days, for a total of three times, at the same time point. On the 7th day after laser photocoagulation, semi quantitative analysis of the leakage intensity of CNV was performed using fundus fluorescence angiography (FFA). Left ventricular perfusion was performed using FITC Dextron, and choroidal slices were quickly taken at a specified time and placed under a fluorescence microscope to observe the CNV area.

Precautions during surgery: after general anesthesia, the pupils of mice were fully dilated, during which the cornea was completely covered with sodium hyaluronate or coupling agent for ultrasound. The operation or examination was completed as soon as possible after anesthesia to prevent cataracts from occurring in the crystalline lens of the mice, which would affect the subsequent observation. After anesthesia, the mice should be warmed, for example, in winter, a thermostatic heating blanket can be used. Gauze was used to pad under the mouse head, and the head position was adjusted until the injection plane was clear under the body microscope. The 33 G insulin needle was used to make a hole about 1 mm away from the black-white junction, and the needle was stopped at the original position after a breakthrough of the fingertip is sensed at the moment of making the hole. The hand was kept stable, and the tip of the needle was not shaken inside the mouse eye. The direction of the needle injection of the Hamiliton microinjector was slanting towards the peripheral part of the eye, so that the needle should not be injected too much, which might hurt the crystalline lens. The tip of the needle can be observed under the microscope.

### 2. Streptozotocin-Induced Mouse Model of Chronic Diabetes Mellitus

C57BL/6 mice (male, 3-5 weeks old) were administered with intraperitoneal injection of streptozotocin (STZ) (7.5 mg/mL; S-0130, Sigma Aldrich, St. Louis, MO, USA), fresh dissolved Na Citrate (CAM) buffer (pH: 4.5-4.7; S4641, Sigma) at a dose of 50 mg/kg, once daily, for 5 consecutive days. After one week, blood sugar ≥300 mg/dL was regarded as the onset of diabetes. Only mice with sustained high sugar for 3 weeks were used for subsequent experiments. Diabetes mice did not use insulin intervention. The blood glucose of mice was measured every month to confirm the status of diabetes. Two months after the onset of diabetes, the mice were randomly divided into four groups with 10 mice in each group. Group 1: 1 uL PBS was injected into the eyes; Group 2: 1 uL Ranibizumab (10 μg) was injected into the eyes; Group 3: 1 uL JP1 (40 μg) was injected into the eyes; and Group 4: 1 uL combination drug (Ranibizumab 5 μg+JP1 20 μg) was injected into the eyes. Intraocular injection once a week, a total of 4 times, to mimic the medication frequency of patients with posterior neovascular eye disease in clinical practice (3 times per month initially+PRN injection). One week after the last administration, Evans blue method was used to evaluate the retinal vascular permeability of mice. FITC Dextron was perfused into the left ventricle to observe the retinal vascular leakage, vascular morphology, and vascular density of mice. Immunofluorescence, immunohistochemistry, and immunoblotting experiments were used to detect the distribution and content of target proteins.

### 3. Fluorescein Fundus Angiography

After sufficient anesthesia, mice were intraperitoneally injected with sodium fluorescein (10%, 0.1 mL/kg). Eye drops Tropicamide were used to dilate the pupils and the degree and area of leakage of choroidal neovascularization (CNV) was evaluated through fluorescence fundus angiography. The leakage intensity of CNV lesions was graded according to the following criteria: 0 (no leakage), weak high fluorescence, or spot fluorescence of no leakage; 1 (suspected leakage), no progressive size of the lesion, or high fluorescence with increased intensity; 2A (leakage), increased high fluorescence intensity, but size does not increase; 2B (significant pathological leakage), increased high fluorescence intensity and size.

4. Stretched Preparation of Choroid after FITC-Dextron Perfusion

Seven days after laser photocoagulation, CNV mice were anesthetized systemically, and 0.2 mL of 5 mg/mL fluorescein-labeled dextran (FITC-dextran, average molecular weight 2×10⁶) was perfused with 34 G insulin needles.

The mice limbs were fixed on a foam board with pins, and the abdomens were kept in a flat state. The skin of the anterior region of the heart was cut, and the hair was removed, and then the muscle layer was cut open using ophthalmic scissors and the chest wall was exposed. At this point, close to where the mouse heart beat was most pronounced, corneal clippers were inserted into the intercostal space, and a piece of the rib was quickly clipped just enough to expose the apex of the heart. The chest wall was pushed forward slightly to expose the apical part of the heart. Insulin needle was inserted quickly into the most obvious place of the apical beat, and after feeling the breakthrough of the needle tip, FITC-dextran was injected into the left ventricle quickly. Thereafter, a piece of gelatin sponge was moistened with saline and placed at the defective rib. 3 min later, CNV mice were fixed with 1% PFA at room temperature for 1-2 h. The conjunctival tissue of the murine eyes was carefully trimmed using corneal scissors. Uniform radial incisions were made so that the choroid-sclera complex of the CNV mouse was placed on a highly adhesive coverslip with the sclera facing downward. A drop of anti-fluorescence quencher was added, flattened using a coverslip, and placed in a wet box. All the procedures were in the dark conditions. The CNV area was measured by fluorescence microscopy (BX53) using Image-Pro Plus 6.0 software (Media Cybernetics, Silver Spring, MD, USA). If the mice had a weak heartbeat midway through the procedure, manual cardiac compression could be considered to ensure that the mice's eyes were sufficiently filled with FITC-dextran and to maintain their vital signs. All procedures were minimally invasive, rapid, and closely monitored throughout the procedure.

5. Stretched Preparation of Retina after FITC-Dextron Perfusion

After the mice were anesthetized, 1 mL of PBS containing 40 mg/mL fluorescein isothiocyanate-glucan was perfused into the left ventricle (average mol wt: 2×10⁶, Sigma, St Louis, MO, USA). After 5 minutes, the eyes were enucleated and fixed overnight in 4% paraformaldehyde. The cornea was excised under an anatomical microscope, and the retina was cut radially from the edge to the equator, completely peeling off the retina. Stretched preparation was made, and observe the flat bracket using a fluorescence microscope and take photos. The vascular density of capillary networks was analyzed using Angio Tool image analysis software. When performing retinal pavement, gelatin sponges may be utilized. After sufficiently wetted with PBS, the eyes were placed on a wet gelatin sponge. The conjunctival tissue of the mouse eye was trimmed using corneal scissors. A 15-degree scalpel was then used to make a break in the mouse eye, followed by insertion of corneal scissors into the break, and the rounded cornea along the corneal rim of the mouse eye was cut. The cornea was cut sufficiently for the subsequent operations. The spherical lens was gently removed in PBS or NS using a water-filled curved needle. The vitreous cavity was washed using a water-filled curved needle and four radial incisions were made in a symmetrical position, centered on the optic nerve, along with the retina and choroid. Next, still in water, the retina and choroid were separated using water and the blunt nature of the curved needle, and stirring the water during the process, without using tweezers to clamp the retina throughout the process.

After complete separation of the fundus retina from the choroid, the retina was slowly unfolded in water, and a curved needle was used to knock out the water, directing the retina to a highly adherent coverslip. The retina was then slowly unfolded on the coverslip, and the excess liquid on the slide was sucked off with a soft paper, and then a drop of antifluorescent quencher was placed on the coverslip. The coverslip was tilted, contacted the one side of the slide, with a motion of tilting-lowering-lying. The coverslip was placed on the retina, with air bubbles removed, to flatten the retina. Do not make both sides of the coverslip touch the slide at the same time, which is not conducive to exhausting the air and flattening the retina.

### 6. H&E Staining

6.1) Sampling: fresh tissues were fixed with 4% paraformaldehyde for at least 24 h. The tissues were trimmed, and placed flat into a dehydrating box.
6.2) Dehydration and wax immersion: the dehydrated box was loaded into a dehydrator, dehydrated with gradient alcohol, and then immersed in 65° C. paraffin wax.
6.3) Embedding: all the tissues were embedded with an embedding machine, cooled down on a −20° C. freezing table, and taken out after the wax was solidified.
6.4) Slicing: the wax was sliced using a paraffin slicer to a thickness of 4 μm; the slices were floated on a slide spreader in warm water at 40° C., then lifted up on slides and baked in a 60° C. oven.
6.5) Deparaffinization: the slices were dewaxed according to standardized procedures.
6.6) Hematoxylin staining: the slices were stained with Hematoxylin staining solution for about 10 min, washed with running water for 2 min and then differentiated with a differentiation solution, then washed with running water for 2 min, stained with a blue promoting solution and then rinsed with running water for 2 min.
6.7) Eosin staining: the slices were dehydrated with 85% alcohol and 95% alcohol for 5 min each, and finally stained with Eosin for 10 min.
6.8) Dewatering and blocking: the slices were placed in anhydrous ethanol I, anhydrous ethanol II, anhydrous ethanol III, xylene I and xylene II for 5 min each, and then blocked with neutral gum.
6.9) Scanning and image analysis.

Precautions: when extracting mouse eyeballs, it is important to avoid pulling the optic nerve. After ophthalmic scissors were inserted into the eyeball, the soft tissue surrounding the optic nerve and optic nerve should be cut short. It is recommended to operate on the same plane as much as possible. After cutting short the tissue on the same plane, the eyeball should be removed. Before sampling, a small incision was made in the left atrial appendage of the mouse (which can be directly made with a fifteen-degree corneal puncture knife, with a neat incision, which will do minimal damage to surrounding tissues). Then, physiological saline was continuously injected into the left ventricle until all the outflow from the right atrial appendage was transparent physiological saline. Fresh paraformaldehyde solution was then used to perfuse the left ventricle. During the perfusion process, muscle twitching could be seen throughout the mouse body, and the tail of the mouse was raised, inject until the mouse's body was stiff, then the sample was taken.

### 7. Immunofluorescence Experiment

### 7.1 Immunofluorescence of Paraffin Slices

1) Place the slices in xylene I for 15 minutes-xylene II for 15 minutes-anhydrous ethanol I for 5 minutes-anhydrous ethanol II for 5 minutes-85% alcohol for 5 minutes-75% alcohol for 5 minutes-double distilled water for 5 minutes.
2) Place the slices in a ceramic jar loaded with EDTA antigen repair buffer (pH 8.0), boil for 30 minutes, and then cool to room temperature for antigen repair. Be careful to ensure that the slices were immersed in the buffer. Cooling, then wash the glass slide in PBS three times, 5 minutes per time.
3) Slightly shake the slices for remove of water, circle the tissue to be observed with a histological pen, then drop goat serum and block for 30 minutes.
4) Gently shake off the blocking solution and rinse three times with a shaking table (washed with PBS), preferably for 5 minutes per time. Then, add the diluted primary antibody to the circle and incubate overnight at 4° C. in a wet box.
5) Rinse the glass slide in PBS three times, about 5 minutes per time. After slightly drying, add secondary antibodies to the circle and incubate at room temperature in the dark for 1 hour.
6) Rinse the glass slide in PBS three times, about 5 minutes per time. Add DAPI staining solution to the circle and incubate at room temperature in the dark for 10 minutes.
7) Dry the slices and block them with anti-fluorescence quenching blocking agent.
8) Observing and collecting images under a fluorescence microscope.

### 7.2 Cellular Immunofluorescence Assay

1) Cell fixation: cell suspension was centrifuged at 2800 rpm and 4° C. for 5 minutes. The supernatant was discarded and 2 mL of 4% paraformaldehyde was added according to the amount of cells precipitated at the bottom for fixation. If cell precipitate was not visible under naked eyes, the cell suspension was centrifuged at 3000 rpm and 4° C. for 10 minutes.
2) Smear preparation: the fixed cell suspension was centrifuged at 2800 rpm and 25° C. for 5 minutes. The supernatant was discarded and PBS was added based on the precipitate at the bottom. A small circle was drawn with a histological pen, the cell suspension was centrifuged at 3000 rpm and at 25° C. for 10 minutes, and 0.5 mL of PBS was added. After mixing, 200 uL of a solution was taken and dropped into the small circle.
3) Cell fixation and membrane rupture: at room temperature, add 50-100 uL of a fixed solution to the circle, after 20 minutes, wash off the fixed solution, add a membrane breaking solution, incubate for 20 minutes, wash three times with PBS, 5 minutes per time.
4) Blocking: block with goat serum at room temperature for 1 hour.
5) Primary antibody incubation: Shake the bed, wash with PBS for 3 to 5 minutes, add pre-prepared primary antibodies to completely cover the cells, and incubate overnight at 4° C. in a wet box.
6) Secondary antibody incubation: remove the primary antibody and wash with PBS for 3 to 5 minutes. Add secondary antibody to cover the cells and incubate at room temperature for 1 hour.
7) DAPI staining: wash with PBS three times, each time for 5 minutes, to remove excess secondary antibodies, then add DAPI staining solution to cover the cells and incubate at room temperature in a dark place for 5 minutes.
8) Microscopic observation: observe and take photos under a fluorescence microscope.

### 8. Immunohistochemical Experiment

8.1) deparaffinization of paraffin slices: the slices were placed in xylene I for 15 min, xylene II for 15 min, xylene III for 15 min, anhydrous ethanol I for 5 min, anhydrous ethanol II for 5 min, 85% alcohol for 5 min, 75% alcohol for 5 min, and then washed with distilled water.
8.2) Antigen repair: the tissue slices were placed in a repair box filled with citric acid antigen repair buffer (pH 6.0), and the antigen repair was carried out in a microwave oven, with medium heat for 8 min until boiling, stopping for 8 min and then turning to medium-low heat for 7 min.
8.3) The slides were cooled, and washed in PBS on a shaker three times, each time for 5 min.
8.4) Block endogenous peroxidase: the slides were placed in 3% hydrogen peroxide solution for 25 min at room temperature, protected from light.
8.5) The slides were placed in PBS and washed 3 times, each for 5 min on a shaker.
8.6) Serum blocking: 3% BSA was added dropwise into the circle and blocked at room temperature for 30 min.
8.7) Addition of primary antibody: shake off the blocking solution, add a primary antibody diluent on the slides, and place the slides flat in a wet box at 4° C. overnight.
8.8) Addition of secondary antibody: wash the slides in PBS three times on a shaker, each for 5 min. Dry the slides and add a secondary antibody dropwise in the circle and incubate at room temperature for 50 min.
8.9) DAB color development: wash the slides in PBS three times, each for 5 min. After the slides were dried, DAB color development solution was added dropwise in a circle, observe the time of color development under the microscope. The process was terminated by rinsing the slices with tap water, and the positive color development was brownish-yellow.
8.10) Re-staining of nuclei: re-stain with hematoxylin for 3 min, wash with tap water, differentiate with hematoxylin differentiation solution for several seconds, rinse with tap water, return blue with hematoxylin solution, and then rinse with running water.
8.11) Dewatering and blocking: the slices were dehydrated in 75% alcohol for 5 min, 85% alcohol for 5 min, anhydrous ethanol I for 5 min, anhydrous ethanol II for 5 min, n-butanol for 5 min, and xylene I for 5 min. After the slices were transparent, take the slices out of the xylene and dry, and then block the slices with neutral gum. Microscopic examination, image acquisition and data analysis.

### 9. Reactive Oxygen Species (ROS) Detection of CNV Tissue Slices

After 5 days of CNV induction, the eyeball was taken, placed in (O.C.T) compound (4583, SAKURA) and immediately frozen. Frozen slices (10 μm thick) of retinal pigment epithelium (RPE)-choroid were made. The slices were stained with diluted fluorescent probe 2′,7′-dichlorodihydrofluorescein diacetate hemin difluoride DCFHDA (HR7814, Biobrab) for ROS detection. ROS levels in retina-choroid slices were measured using fluorescence microscopy.

### 10. Determination of MDA, SOD, GPx

Glutathione peroxidase (GPx), malondialdehyde (MDA), and superoxide dismutase (SOD) were detected of choroidal tissues using kits. The protein concentrations were detected using the BCA protein assay kit (Jiangsu Bitai Institute of Biotechnology, China). All experiments were repeated five times.

### 11. Measurement of Intracellular ROS

Intracellular ROS levels were detected using a reactive oxygen species detection kit (Jiangsu Beyotime Institute of Biotechnology, China). Cells were incubated with DCFH-DA (Jiangsu Beyotime Institute of Biotechnology, China) at 37° C. for 20 min, and washed with serum-free medium. The cells were observed under a fluorescence microscope.

### 12. Immunoblotting Experiments (Western Blotting)

Extraction of total protein from cells or tissues: all operations were performed on ice; first, the cells were washed slowly with PBS for 2 times, and the culture fluid was removed. RIPA lysate was added to the dish, blown uniformly, and shaken at 4° C. for 30 min. Generally, if the six-well plate is full of cell seeding and the cell morphology is small and dense, 100 uL/well can be added, and if the cells are only full to 60%-80%, 60-80 uL/well can be added. The solution was centrifuged at 4° C., 12,000×g, for 15 min and the supernatant was collected to measure the concentration of the proteins. The standard product was BSA, and the dilution solution of the standard product was saline. BCA protein concentration was determined as follows: BCA reagent A: BCA reagent B (Biyun Tian) (50:1) were used to prepare the appropriate amount of BCA working solution. For example: 4 mL of BCA reagent A+80 uL of BCA reagent B; 10 uL of BSA (5 mg/mL) was diluted to 100 uL with saline dilution, so that the final concentration was 0.5 mg/mL. 0, 1, 2, 4, 8, 12, 16, 20 uL of the standard products were added to a 96-well plate, and the dilution solution of the standard product was further added to reach 20 uL (at this time, the concentration was 0, 0.5, 1, 2, 4, 6, 8, 10, respectively); appropriate volume of sample (1 uL) was added to the 96-well plate, and the dilution solution of the standard product was further added to reach 20 uL. 200 uL of BCA working solution was added to each well, and rested at 37° C. for 30 min; the absorbance value at 570 nm was measured. The protein concentration was calculated according to the standard curve. Protein intake: the lowest protein concentration of a group of proteins was selected as the basis, and 30 μg/15 uL of total protein was taken, and the remaining volume was made up with double-distilled water ddH₂O. The mixture was boiled at 100° C. for 5 min and stored at 4° ° C. 70-80 μg of sample was added to each lane (≤30 uL for 15 wells, ≤50 uL for 10 wells), and the total amount of sample protein in each lane was equal. 30 μg/20 uL corresponded to 15 uL/lane first. Turn on the electrode, 80 V for 30-45 min for the upper gel, 110 V for the lower gel, and stop electrophoresis when the bromophenol blue indicator migrated to the downstream edge of the separator gel. The electrophoresis can be started at 90 V, and then changed to 120 V after the molecular weight of the marker was basically separated. Corresponding size of PVDF membrane (8.3 cm×5.2 cm, pre-soaked in methanol for 60 s) and the lower gel layer were immersed in a transfer buffer for equilibration for 15-30 min, and then the sponge, filter paper, gel, PVDF membrane, filter paper, and sponge were arranged in the order from the bottom to the top through a sandwich method. The air bubbles between the membrane and the gel were removed, and the water was excluded. The electrophoresis was performed in a constant current mode, 0.22A, 90 min, and the voltage was >110v. Blocking: the blocking solution was 1×TBST+5% (5 g/100 ml mass/volume ratio, the same below) skimmed milk powder, and the nitrocellulose membrane was put into the blocking solution, placed on a shaking bed for 1-2 h at room temperature, and washed with PBST (TBST) 3 times, each time for 5 min. Primary antibody blocking: the primary antibody was diluted to 1:1000 with an antigen diluent and placed on a shaking table, 60 times/min, overnight at 4° C., washed with PBST (TBST) for 5 min×5 times (2 times is acceptable). Secondary antibody blocking: the secondary antibody was diluted to 1:1000 with 1×TBST+5% skimmed milk powder. Sheep anti-rabbit IgG-HRP or sheep anti-mouse IgG-HRP was incubated on a shaker at room temperature for 1 h, washed with PBST (TBST) for 3 times, each time for 15 min. The developing solution immobilon 1:1 was prepared, the developing solution was evenly applied to the membrane and exposed.

### 13. Human Umbilical Vein Endothelial Cells (HUVECs) Tube-Formation

HUVECs (2.5×10⁵) were suspended in 250 uL of serum-free DMEM and implanted into the top chamber of a 24-well transwell plate (Corning Inc., Corning, NY). The bottom chamber of the transwell plate was filled with 600 uL of DMEM containing 10% fetal bovine serum. 48 h later, the cells were stained with methanol and 0.1% crystalline violet, imaged and counted with an Olympus IX70 inverted microscope (Tokyo, Japan). The average cell number of the four stained membrane images was obtained using ImageJ software (NIH, Bethesda, MD). Each experiment was repeated three times.

### 14. EdU Experiment

1) The cell culture medium was completely diluted and prepared into an EdU solution, with a dilution ratio of 1000:1. 50 μM EdU medium was prepared.
2) 100 uL of 50 μM EdU medium was added to each well and incubated for 2 h.
3. The cell surface was gently washed with PBS for 2 times, each for 5 min.
4) 50 uL cell fixative (i.e., PBS containing 4% paraformaldehyde) was added to each well and incubated for 30 min at room temperature.
5) 50 uL of 2 mg/mL glycine added to each well and incubated on a decolorization shaker for 5 minutes, and the glycine solution was discarded.
6) 100 uL PBS added to each well for cleaning on a decolorization shaker for 5 minutes, and PBS discarded.
7) 100 uL of a penetrant (0.5% PBS of TritonX-100) was added to each well and incubated on a decolorization shaker for 10 minutes, cleaned with PBS for 5 minutes.
8) 100 uL of 1× Apollo of L® dyeing reaction solution was added to each well and incubated at room temperature on a decolorization shaker without light for 30 minutes.
9) 100 uL penetrant (PBS of 0.5% TritonX-100) was added to each well and cleaned in a decolorization shaking table for 3 times, each time for 10 minutes.
10) 100 uL of methanol was added to each well for cleaning 2 times, each time for 5 minutes, and then cleaned with PBS for 5 minutes.
11) An appropriate amount of 1× Hoechst33342 reaction solution was prepared with deionized water and stored in a dark place.
12) 100 uL of 1× Hoechst 33342 reaction solution was added to each well, incubated at room temperature on a decolorization shaker without light for 30 minutes.
13) 100 uL of PBS was added to each hole for cleaning 1-3 times; testing was carried out as soon as possible after dyeing; the sample was stored in a dark wet box at 4° C. not longer than 3 days. Climbing or smearing cells can be quenched with an anti-fluorescence agent, sealed with a sealing agent, and stored at 4° ° C. for testing.

### 15. Transwell Experiment

1) The 24-well plate and Transwell were prepared the day before the test. FN with a master concentration of 1 mg/mL was diluted 10-fold to a final concentration of 100 μg/mL. 50 uL of FN was applied to the bottom of the chambers, air-dried for 2 h on an ultra-clean bench, and incubated in a cell culture incubator overnight.
2) On the day of the test, the cells were digested with 0.25% trypsin, counted and the cell density was adjusted to 1×10⁶ cells/mL; 100 uL of cells were seeded into the upper layer of the Transwell, and 600 uL of culture medium with 10% FBS was added outside the chamber.
3) After 12 hours of incubation, the Transwell was taken out and the cells were fixed in 95% methanol for 20 min.
4) The cells were washed with PBS for three times, each for 5 min, and stained with 0.4% Taipan blue for 30 min.
5) The cells were washed with PBS for three times, each for 5 min, and the cells in the upper chamber were wiped off with a cotton swab.
6) The small chamber was placed on the slide and pictures were taken under microscope.

### 16. Research on the Distribution of FITC-JP1

One week after laser induction, mice with choroidal neovascularization were randomly divided into two groups (10 mice per group), and injected intraperitoneally with 5 mg of FITC-JP1 and 0.99 mg of FITC 100 uL (both groups were injected with equal amounts of FITC intraperitoneally), respectively. The intensity of FITC in the living retina was determined using the FFA method (n=5/group). To evaluate the accumulation of FITC in CNV lesions, mice were euthanized at 1, 3, 8, 24, and 48 h after intraperitoneal injection of FITC-JP1 or FITC, respectively. Then, choroidal spreads were observed by fluorescence microscopy.

### 17. Evans Blue Test

Blood retinal barrier (BRB) was quantified using the Evan Blue method, as described previously with minor modifications.

Evans blue (45 mg/kg) was injected through the tail vein of mice for more than 10 s. The mice were then placed on a warm pad for 2 h. Plasma Evans blue concentration was determined by taking 100 uL of blood. The thoracic cavity of the mice was opened, and the left ventricle was perfused with 0.05 M, pH 3.5 citrate buffer at 37° C. for 2 min to clear the dye from the blood vessels. Next, both eyes were removed and bisected along the equator. Retinas were dissected under a stereomicroscope and dried at 70° C. for 24 h. Each sample was incubated with 130 uL formamide (Sigma) at 70° ° C. for 18 h. Evans blue dye bound to serum albumin was extracted from the retina. Extracts were centrifuged at 4° C., 65,000 rpm for 60 min. The blood samples were centrifuged at 4° C., 12,000 rpm for 15 min and diluted to 1/100 with formamide prior to spectral evaluation. To evaluate the Evans blue concentration, the absorbance of the retinal extracts and plasma samples was measured at 620 nm and compared with the standard curve. The BRB is calculated using the following formula, and the result is expressed with microliters plasma×gram retina dry wt⁻¹·hour⁻¹:

$$\frac{Evans blue ( μ g ) / Retina dry weight ( g )}{\begin{matrix}Evans blue concentration ( μ g ) / \\ Plasma ( μ L ) \times Circulation time ( hour )\end{matrix}}$$

### 18. Statistical Analysis

All data are expressed as mean±standard error of the mean (SEM). Comparisons between groups were made using Student's t test, while comparisons between three and more groups were made using one-way ANOVA. Statistical differences were defined by a P value of <0.05. Statistical analysis was performed using GraphPad Prism v9.0 software (GraphPad software, Inc., La Jolla, CA, USA).

It will be obvious to those skilled in the art that changes and modifications may be made, and therefore, the aim in the appended claims is to cover all such changes and modifications.

What is claimed is:

1. A method for treating or preventing a neovascular eye disease comprising administering a patient in need thereof a pharmaceutical composition comprising a polypeptide, the peptide having an amino acid sequence I or II:
I:
FPGSDRF (SEQ ID NO: 1)-Z;
II:
X-FPGSDRF (SEQ ID NO: 1)-Z;
wherein:
S represents phosphorylated serine;
X and Z independently represents an amino acid or an amino acid sequence;
X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉ (SEQ ID NO: 2), 6-aminohexanoic acid-(R)₉-F (SEQ ID NO: 3); and
Z is selected from (G)ₙ-RGD or A-(G)ₙ-RGD (SEQ ID NO: 4), where n is an integer greater than or equal to 0, in the range of 0-10.

2. The method of claim 1, wherein the neovascular eye disease is wet macular degeneration.

3. The method of claim 1, wherein the neovascular eye disease is neovascular age-related macular degeneration (nAMD).

4. The method of claim 1, wherein the neovascular eye disease is diabetic retinopathy (DR).

5. The method of claim 1, wherein the neovascular eye disease is retinal vein occlusion (RVO), neovascular glaucoma, or retinopathy of prematurity (ROP).

6. The method of claim 1, wherein the polypeptide comprises an acetylated N-terminal and an amidated C-terminal.

7. The method of claim 1, wherein the polypeptide has an amino acid sequence of FPGSDRF-RGD, and serine (S) of the amino acid sequence of FPGSDRF-RGD is phosphorylated.

8. The method of claim 1, wherein the pharmaceutical composition further comprises a pharmaceutically acceptable excipient.

9. The method of claim 1, wherein the pharmaceutical composition is for intraocular injection or eye drops.

10. The method of claim 1, wherein the pharmaceutical composition is for extraocular administration.


SEQUENCE LISTING (27 sequences)

SEQ ID NO: 1 - protein, 7 aa, synthetic construct
FPGSDRF

SEQ ID NO: 2 - protein, 9 aa, synthetic construct
RRRRRRRRR

SEQ ID NO: 3 - protein, 10 aa, synthetic construct
RRRRRRRRRF

SEQ ID NO: 4 - protein, 5 aa, synthetic construct
  REPEAT 2
AGRGD

SEQ ID NO: 5 - protein, 11 aa, synthetic construct
FPGSDRFGRGD

SEQ ID NO: 6 - protein, 14 aa, synthetic construct
FPGSDRFGGGGRGD

SEQ ID NO: 7 - protein, 20 aa, synthetic construct
FPGSDRFGGGGGGGGGGRGD

SEQ ID NO: 8 - protein, 11 aa, synthetic construct
FPGSDRFARGD

SEQ ID NO: 9 - protein, 12 aa, synthetic construct
FPGSDRFAGRGD

SEQ ID NO: 10 - protein, 15 aa, synthetic construct
FPGSDRFAGGGGRGD

SEQ ID NO: 11 - protein, 21 aa, synthetic construct
FPGSDRFAGGGGGGGGGGRGD

SEQ ID NO: 12 - protein, 11 aa, synthetic construct
FFPGSDRFRGD

SEQ ID NO: 13 - protein, 12 aa, synthetic construct
FFPGSDRFGRGD

SEQ ID NO: 14 - protein, 15 aa, synthetic construct
FFPGSDRFGGGGRGD

SEQ ID NO: 15 - protein, 21 aa, synthetic construct
FFPGSDRFGGGGGGGGGGRGD

SEQ ID NO: 16 - protein, 12 aa, synthetic construct
FFPGSDRFARGD

SEQ ID NO: 17 - protein, 13 aa, synthetic construct
FFPGSDRFAGRGD

SEQ ID NO: 18 - protein, 22 aa, synthetic construct
FFPGSDRFAGGGGGGGGGGRGD

SEQ ID NO: 19 - protein, 19 aa, synthetic construct
RRRRRRRRRFPGSDRFRGD

SEQ ID NO: 20 - protein, 29 aa, synthetic construct
RRRRRRRRRFPGSDRFGGGGGGGGGGRGD

SEQ ID NO: 21 - protein, 20 aa, synthetic construct
RRRRRRRRRFPGSDRFARGD

SEQ ID NO: 22 - protein, 30 aa, synthetic construct
RRRRRRRRRFPGSDRFAGGGGGGGGGGRGD

SEQ ID NO: 23 - protein, 20 aa, synthetic construct
RRRRRRRRRFFPGSDRFRGD

SEQ ID NO: 24 - protein, 30 aa, synthetic construct
RRRRRRRRRFFPGSDRFGGGGGGGGGGRGD

SEQ ID NO: 25 - protein, 21 aa, synthetic construct
RRRRRRRRRFFPGSDRFARGD

SEQ ID NO: 26 - protein, 31 aa, synthetic construct
RRRRRRRRRFFPGSDRFAGGGGGGGGGGRGD

SEQ ID NO: 27 - protein, 10 aa, synthetic construct
FPGSDRFRGD